(19) United States
(12) Patent Application Publication
(10) Pub. No.: US 2023/0329666 A1
(43) Pub. Date: Oct. 19, 2023

(54) DETECTING AND DE-NOISING ABNORMAL LUNG SOUNDS

(71) Applicant: Sonavi Labs, Inc., Baltimore, MD (US)

(72) Inventor: Ian Mitra McLANE, Baltimore, MD (US)

(21) Appl. No.: 17/659,205

(22) Filed: Apr. 14, 2022

(51) Int. Cl. A61B 7/04; A61B 7/00; A61B 5/00; A61B 5/08

(52) U.S. Cl. CPC A61B 7/04; A61B 7/003; A61B 5/7203; A61B 5/726; A61B 5/08

(57) ABSTRACT

Aspects disclosed herein disclose a system and method for improving digital stethoscopes and their application and operation. A first method de-noises an auditory signal. A second decomposes an auditory signal into sub-components. A third method extracts a respiratory cycle from the auditory signal. A fourth method counts respiratory abnormalities based on the respiratory cycle.

### TECHNICAL FIELD

Aspects relate generally to detecting a respiratory abnormality and a method of operation thereof.

### BACKGROUND

Patients with respiratory illnesses such as cystic fibrosis need regular monitoring of their lungs in order to monitor disease progression. Conventionally, this monitoring involves obtaining frequent computerized tomography (CT) scans of the patient's lungs. This poses a problem because CT scans use X-rays to generate images of the patient's lungs. Frequent exposure to X-rays, however, can be harmful to patients because X-rays expose patients to radiation. Over time, X-rays can accumulate in a patient's body, which can have harmful health implications. Thus, systems and methods are needed to monitor respiratory illnesses without having to perform CT scans as frequently.

The problem can be partially resolved using digital auscultation to monitor a patient's lungs. Digital auscultation is a well-known method for assessing lung sounds. Using digital auscultation, a patient's lung sounds can be monitored by recording the lung sounds, so that these sounds can be assessed to determine if any abnormal lung sounds can be heard. These sounds can be, for example, crackling sounds in the patient's lungs during inhalation and exhalation that can indicate severity of a respiratory disease or indicate progression of a disease.

Assessing lung sounds based on digital auscultation, however, remains a subjective process. In conventional practice, interpreting lung sounds still relies on human interpretation. For example, while lung sounds can be recorded using digital stethoscopes, these lung sounds must still be analyzed by highly trained doctors, so the doctor can determine if any abnormal lung sounds are present. This is difficult, however, because these lung sounds are often noisy due to external noise, such as motion artifacts, background noise, etc. This noise can interfere with the detection of important signals indicating the abnormal lung sounds, which are the important signals (e.g., crackle noises in the lungs) correlating to disease progression. Due to these noisy signals, doctors analyzing a patient's lung sounds sometimes have a difficult time detecting important signals and sometimes have to make guesses as to what sounds are, for example, crackle sounds and what sounds are irrelevant noise.

Additionally, because of the limitations of the human ear and the number of crackles that can occur during an inhaling or exhaling, the precise number of times an abnormal lung sound occurs may be hard to detect. For example, a typical cystic fibrosis patient can have up to 15 crackles occur during an inhalation or exhalation. A doctor typically cannot count all instances of these crackles because the human ear is not capable of recognizing this many crackling sounds in such a short period. Thus, doctors, rather than detect the precise number of crackles, only listen for fingerprints or artifacts indicating crackles are happening. Without tools and methods that are more precise, doctors cannot know at a granular level how many crackles actually occur during any given inhalation or exhalation merely by listening to recordings of lung sounds. This information, however, is useful to know because it is relevant to assessing the severity of disease progression. Thus, systems and methods are needed to better de-noise lung sounds obtained via digital auscultation and to more precisely detect important noise signals that are correlated to disease progression.

### SUMMARY

Aspects disclosed herein provide improved systems and methods for detecting and de-noising lung sounds obtained via digital auscultation. The systems and methods also allow health care providers to more precisely detect important noise signals that are correlated to disease progression. For example, using the systems and methods disclosed herein, abnormal lung sounds, such as crackles in a patient's lungs can be detected, and the precise number of crackles during an inhalation and exhalation can be determined.

The systems and methods provide significant advantages over conventional systems because conventional systems do not allow for such precise detection and measurement of lung sounds indicative of abnormalities. Moreover, the systems and methods allow for improved ways of monitoring respiratory disease progression without the need for patients to obtain frequent CT scans, as is typically the case today for diseases such as cystic fibrosis. As a result, the amount of radiation that patients are exposed to is significantly reduced. This reduction in exposure to radiation is a significant improvement in the way patients are treated.

In a first aspect, a computer-implemented system and method for de-noising an auditory signal is disclosed. The system can implement a method to partition an auditory spectrogram representing the auditory signal into a plurality of windows of equal length timeframes, where each of the windows indicates a frequency response of the auditory signal within each of the timeframes. The auditory signal can represent a lung sound. Each of the windows can be processed using a neural network trained to remove unwanted noise signals from the auditory signal. In aspects, the processing can include: (i) identifying an odd number of consecutive windows, (ii) identifying a middle window from the odd number of consecutive windows, where the middle window is a window to have the unwanted noise signals removed, (iii) identifying an even number of windows preceding the middle window, (iv) identifying an even number of windows following the middle window, (v) inputting the middle window, the even number of windows preceding the middle window, and the even number of windows following the middle window into the neural network, and (vi) computing, using the neural network, a vector representing the auditory signal with the unwanted noise signals removed.

In a second aspect, a computer-implemented system and method for decomposing an auditory signal into sub-components is disclosed. The system can implement a method to filter the auditory signal by performing a wavelet transform, where the wavelet transform utilizes a wavelet representing a sound indicating a respiratory abnormality. The wavelet transform extracts a signal from the auditory signal indicating the respiratory abnormality. In aspects, the system and method can determine whether a signal amplitude for the extracted signal is above a predetermined threshold value. Based on determining the signal amplitude is above the predetermined threshold value, the extracted signal can be stored as an instance of the respiratory abnormality. Based on determining the signal amplitude is below the predetermined threshold value, the extracted signal can be stored as an instance indicating no respiratory abnormality. In aspects, the amplitude or width of the wavelet can be adjusted and the aforementioned processes can be performed using the amplitude adjusted or width adjusted wavelet. The purpose of doing this is to capture any variations of the sound indicating the respiratory abnormality.

In a third aspect, a system and method for extracting a respiratory cycle is disclosed. The system can implement a method to receive an auditory signal representing a vesicular sound. The vesicular sound refers to a patients breathing with sub-components representing a respiratory abnormality removed. The removal of the sub-components indicating a respiratory abnormality can be done using the systems and methods described with respect to the second aspect described above. In aspects, the method can further partition the auditory signal into segments. In aspects, a transformation to each of the segments can be applied to determine a signal envelope. In aspects, a moving average window to the signal envelope can be applied to obtain an averaged signal envelope. Alternatively, in aspects, a transformation can be applied to each of the segments to obtain a frequency response of the auditory signal within each of the segments. The frequency response can be summed across the segments to obtain a summed frequency response. An inverse transformation can be applied to the summed frequency response to obtain an averaged signal envelope.

In aspects, once an averaged signal envelope is obtained, a point where the averaged signal envelope initially has an amplitude greater than a threshold value can be identified. A mean value for the amplitude of the averaged signal envelope for a period of time after the point can be determined. The method can further determine whether the mean value is greater than twice the threshold value. Based on determining that the mean value is greater than twice the threshold value, the point can be identified as a start of a respiratory cycle. In aspects, a further point where the averaged signal envelope is less than the threshold value can be identified. A further mean value for the amplitude of the averaged signal envelope for a further period of time prior to the further point can be determined. The method can determine whether the further mean value is greater than twice the threshold value. Based on determining the further mean value is greater than twice the threshold value, the further point can be identified as an end of the respiratory cycle. In aspects, a minimum point for the amplitude of the averaged signal envelope between the start of the respiratory cycle and the end of the respiratory cycle can be determined. The minimum point can be identified as a start of an expiration event. In this way, a respiratory cycle can be extracted. The respiratory cycle can be used to determine where in the respiratory cycle respiratory abnormalities such as crackles occur.

In a fourth aspect, a system and method for counting respiratory abnormalities is disclosed. The system can implement a method to receive an auditory signal representing respiratory abnormality sounds. The auditory signal representing the respiratory abnormality sounds can be extracted using the systems and methods described with respect to the second aspect above. The auditory signal can, for example, be an audio signal with only the crackle sub-components. In aspects, the method can determine whether an amplitude for the auditory signal is above an inspiration threshold. The inspiration threshold refers to a threshold value above which the system can determine that the signal represents a crackle during inhalation. Based on determining the amplitude is above the inspiration threshold, an instance of a respiratory abnormality can be identified. In aspects, the method can further determine whether an amplitude for the auditory signal is above an expiration threshold. The expiration threshold refers to a threshold value above which the system can determine that the signal represents a crackle during an exhalation. Based on determining the amplitude is above the expiration threshold, a further instance of the respiratory abnormality can be identified.

Certain aspects of the disclosure have other steps or elements in addition to or in place of those mentioned above. The steps or elements will become apparent to those skilled in the art from a reading of the following detailed description when taken with reference to the accompanying drawings.

### BRIEF DESCRIPTION OF THE DRAWINGS

The accompanying drawings, which are incorporated herein and form a part of the specification, illustrate aspects of the present disclosure and, together with the description, further serve to explain the principles of the disclosure and to enable a person skilled in the arts to make and use the aspects.

FIG. 1 is an exemplary control flow for a system for detecting and de-noising an auditory signal according to aspects.

FIG. 2A is an exemplary control flow for a deep learning de-noising network used to de-noise the auditory signal according to aspects.

FIGS. 2B-2C are exemplary methods for performing the de-noising of the auditory signal using the deep learning and de-noising network according to aspects.

FIG. 3A is an exemplary control flow for how the auditory signal is decomposed to its components using a wavelet packet decomposition process according to aspects.

FIGS. 3B-3D are exemplary methods for decomposing an auditory signal into its components using the wavelet packet decomposition process according to aspects.

FIG. 4A is an exemplary control flow for extracting a respiratory cycle according to aspects.

FIGS. 4B-4E are exemplary methods for extracting the respiratory cycle according to aspects.

FIG. 4F shows an exemplary method for counting respiratory abnormalities according to aspects.

FIG. 5 shows an example digital stethoscope and base station that can be used to implement the functions of the system according to aspects.

FIG. 6 shows an exemplary architecture of the digital stethoscope according to aspects

FIG. 7 shows an exemplary architecture of the base station according to aspects.

FIG. 8 shows exemplary components of the digital stethoscope according to aspects.

FIG. 9 shows exemplary components of the base station according to aspects.

In the drawings, like reference numbers generally indicate identical or similar elements. Additionally, generally, the left-most digit(s) of a reference number identifies the drawing in which the reference number first appears.

### DETAILED DESCRIPTION

Signal Intake, Preprocessing, and Motion Artifact Detection

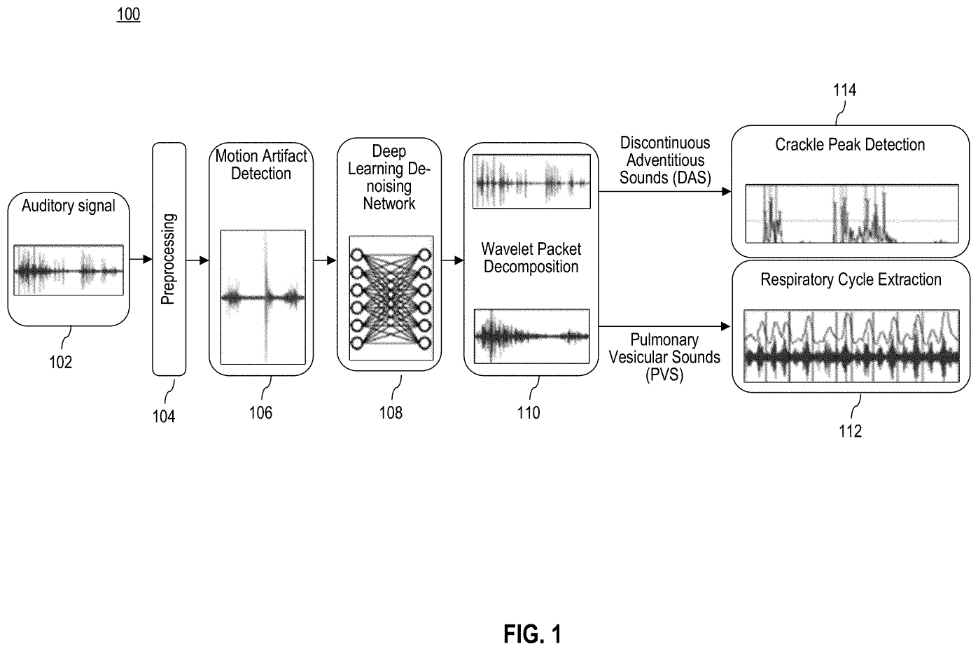

FIG. 1 shows a control flow for a system 100 for detecting and de-noising an auditory signal 102 according to aspects. The auditory signal 102 can be, for example, a recording of a lung sound. In aspects, the auditory signal 102 can be obtained via a digital stethoscope and/or other computing device on which the auditory signal 102 can be recorded and/or stored. The computing device can be, for example, a computer, server, etc. For the purposes of this disclosure, it is assumed that the auditory signal 102 initially received by the system 100 is a raw signal. Thus, the raw signal will likely include unwanted noise signals due to external noise, such as motion artifacts from moving a digital stethoscope, background noise captured by a microphone recording the auditory signal 102, etc. In aspects, the control flow can proceed initially to de-noise the auditory signal 102. As a part of the de-noising, the control flow can proceed to a preprocessing stage 104. The purpose of the preprocessing stage 104 is to put the auditory signal 102 in a form to simplify signal processing at later stages of the control flow, and to remove unnecessary information that is irrelevant to extracting signals indicating respiratory abnormalities and/or is irrelevant to extracting a respiratory cycle.

In aspects, the preprocessing stage 104 can include the auditory signal 102 being low-pass filtered and down-sampled. In aspects, the filtering can be performed using any known signal processing filter, for example, a Butterworth filter, a Chebyshev filter, an Elliptic filter, a Bessel filter, etc. For example, the auditory signal 102 can be low-pass filtered with a fourth-order Butterworth filter at a 4 kHz cutoff. The filtering can remove any unwanted noise signals above the cutoff because typically normal respiratory sounds signals are found between 50-2500 Hz, and sounds representing respiratory abnormalities such as crackles, wheezes, stridor, squawks, or rhonchi exhibit frequency profiles below 4000 Hz. In aspects, the down-sampling can be performed to reduce the size of the auditory signal 102 so that it can be stored in bandwidth limited systems and processed quickly. For example, the auditory signal 102 can be down-sampled from 44.1 kHz to 8 kHz.

In aspects, once the preprocessing stage 104 is performed, and the auditory signal 102 is filtered and down-sampled, control can pass to a motion artifact detection module 106. The motion artifact detection module 106 enables the detection and removal of unwanted noise signals due to motion artifacts. Motion artifacts refer to noise signals generated due to the movement of a sensor or microphone when recording the auditory signal 102. These motion artifacts can occur, for example, when a digital stethoscope that records the auditory signal 102 is moved around due to patient movements. Motion artifacts are characterized by being broadband signals that occur over a short period of time. Therefore, they can be misclassified as an auditory abnormality such as a crackle. In order to detect and remove motion artifacts from the auditory signal 102, an auditory spectrogram representation of the auditory signal 102 is generated. The auditory spectrogram refers to a two dimensional representation of the auditory signal 102 showing the frequencies of a signal as it varies with time. In aspects, to generate the auditory spectrogram, the auditory signal 102 can be partitioned into windows with an overlap. For example, these windows can be 10 millisecond (ms) windows with 90% overlap with each other. These windows can be converted into a frequency domain representation using a Fast Fourier Transformation (FFT) with 265 sample points (i.e., a 256-point FFT).

In aspects, once the auditory spectrogram is obtained, regions of interest are identified that are likely to be signals caused by motion artifacts. In aspects, these regions of interest are identified as those that contain high spectral content above 1 kHz, with a total span greater than 2 kHz. In aspects, a threshold can be defined by the total average energy above 1 kHz of the entire signal. The number of consecutive frequency bands above the threshold quantifies a spectral span. To identify motion artifacts, consecutive frames of 10 to 100 ms exhibiting similar high-energy content are identified as regions that are likely to be motion artifacts. These identified regions are then removed.

### Deep Learning De-Noising Network

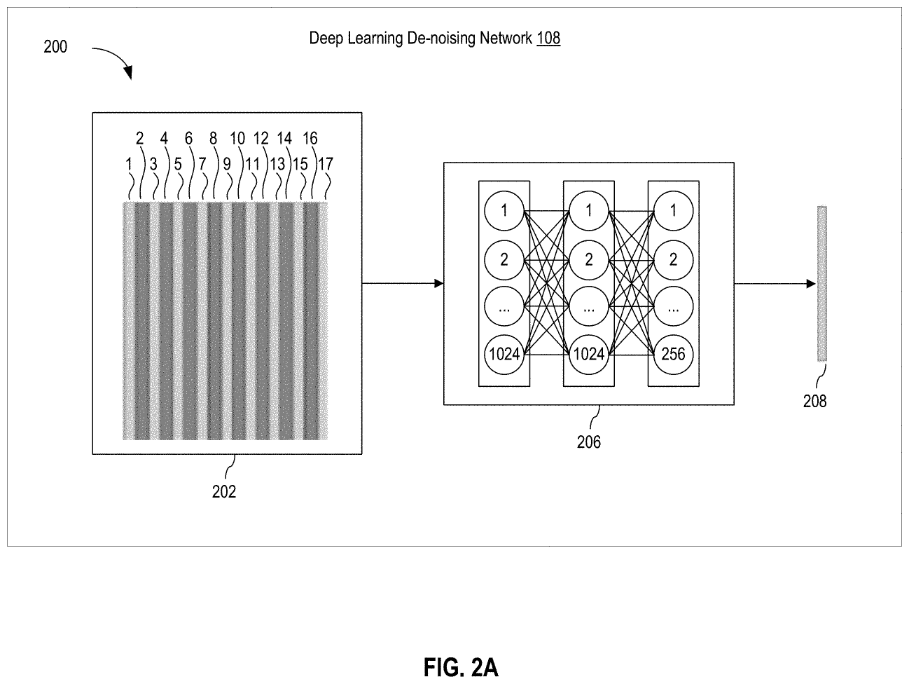

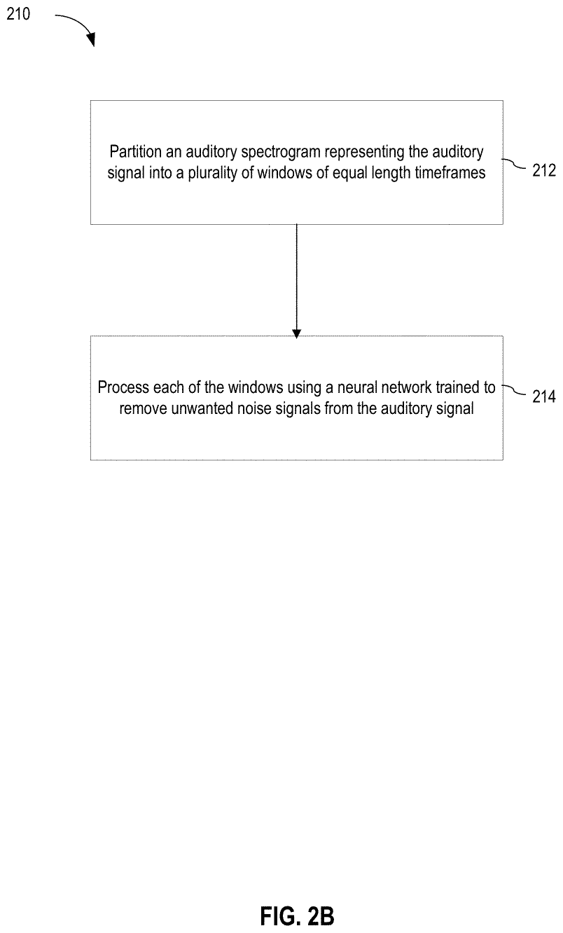

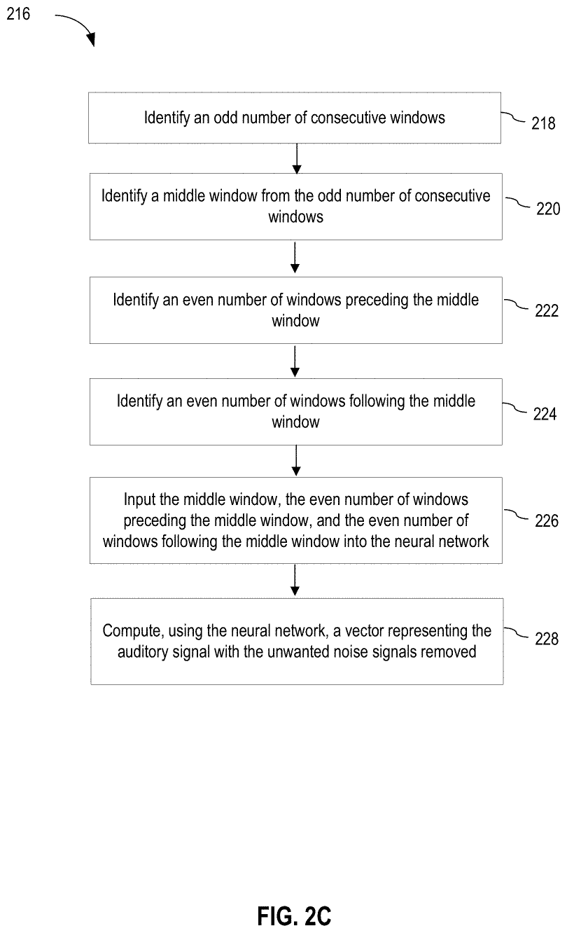

In aspects, once the motion artifact detection module 106 performs its functions and unwanted noise related to motion artifacts is removed, the auditory signal 102 and control can pass to a deep learning and de-noising network 108 to further de-noise the auditory signal 102. The further noise to be removed can be noise related to environmental conditions, such as background noise that is recorded as a part of recording the lung sounds. In aspects, the deep learning and de-noising network 108 can include a neural network 206 (shown in FIG. 2A) that is trained to remove noise related to environmental conditions. The de-noising scheme employed by the deep learning and de-noising network 108 is shown in more detail in FIG. 2A.

FIG. 2A shows the control flow 200 for the deep learning and de-noising network 108 used to de-noise the auditory signal 102 according to aspects. In aspects, control flow 200 can begin by having an auditory spectrogram 202 input into the deep learning and de-noising network 108. The auditory spectrogram 202 can represent the auditory signal 102 resulting from the processing performed by the motion artifact detection module 106. Thus, the auditory spectrogram 202 can be the spectrogram output by the motion artifact detection module 106 with the noise due to the motion artifacts removed.

In aspects, once the auditory spectrogram 202 is received, the deep learning and de-noising network 108 can partition the auditory spectrogram 202 into a plurality of windows of equal length timeframes. In FIG. 2A, these windows are labeled {1, 2, . . . , 17}. The 17 windows are merely exemplary and representative of the number of windows the auditory spectrogram 202 can be partitioned into. There can be more or less windows. Each of these windows can indicate a frequency response of the auditory signal 102 within each of the timeframes. In aspects, the deep learning and de-noising network 108 can begin processing each of the windows to de-noise each by using a neural network 206 trained to remove unwanted noise signals from the auditory signal 102. In aspects, the neural network 206 can be trained using recordings with varying levels of stationary and non-stationary ambient noise in order to train the neural network 206 to detect background/environmental noise and differentiate that noise from lung sounds.

In aspects, once partitioned, each window can be processed by the neural network 206 sequentially. The processing can remove noise from each of the windows that are processed. The general procedure by which the windows are processed is as follows. First, an odd number of consecutive windows is identified. Second, a middle window from the odd number of consecutive windows can be identified, where the middle window is a window to have the unwanted noise signals removed. Third, an even number of windows preceding the middle window can be identified. Fourth, an even number of windows following the middle window can be identified. Fifth, the middle window, the even number of windows preceding the middle window, and the even number of windows following the middle window can be input into the neural network 206. Sixth, the neural network 206 can then proceed to process the input windows to remove noise from the middle window by analyzing the middle window in the context of the surrounding windows to determine what signals in the middle window are likely to be background/environmental noise.

The above described process is the general manner in which the windows are de-noised. However, because the neural network 206 processes each of the windows, special cases need to be handled where the window being processed does not have an even number of windows either preceding it or following it. By way of example, in FIG. 2A, the window labeled “1” does not have an even number of windows preceding it because it is the first window. Similarly, the window labeled “17” does not have an even number of windows following it because it is the last window. In such cases, in order to process the window, it is assumed that there are an even number of windows preceding or following the window, however, their frequency components are set to zero.

In aspects, the output of the processing done by the neural network 206 can be a vector 208 representing the window processed but with the unwanted noise signals removed. In aspects, once the neural network 206 processes all the windows, it can have a full set of windows with their background/environmental noise removed.

In aspects, the architecture of the neural network 206 can have the neural network 206 have three hidden layers of sizes: 1024, 1024, and 256. In aspects, the odd number of consecutive windows can be varied. In a preferred aspect, the odd number of consecutive windows to be processed can equal nine. In aspects, the even number of windows preceding and following the middle window can also be varied. In a preferred aspect, the even number of windows can equal four. In aspects, the odd number of consecutive windows can overlap. This overlap allows the neural network 206 to process the windows by recognizing the continuity between windows and frequency responses therein. By how much the windows overlap can be varied. In a preferred aspect, the windows can overlap with one another by 90%. That is, the preceding window can overlap with the following window, or vice versa, by 90%.

FIGS. 2B and 2C show methods 210 and 216 for performing the de-noising of the auditory signal 102 using the deep learning and de-noising network 108 according to aspects. FIG. 2B shows a two-step process by which the auditory signal 102 is de-noised. At step 212, the auditory spectrogram 202 representing the auditory signal 102 is partitioned into a plurality of windows of equal length timeframes, where each of the windows indicates a frequency response of the auditory signal 102 within each of the timeframes. At step 214, each of the windows is processed using a neural network 206 trained to remove unwanted noise signals from the auditory signal 102.

FIG. 2C is a method 216 for how the neural network 206 processes each of the windows. At step 218, an odd number of consecutive windows is identified. At step 220, a middle window from the odd number of consecutive windows can be identified, where the middle window is a window to have the unwanted noise signals removed. At step 222, an even number of windows preceding the middle window can be identified. At step 224, an even number of windows following the middle window can be identified. At step 226, the middle window, the even number of windows preceding the middle window, and the even number of windows following the middle window can be input into the neural network 206. At step 228, the neural network 206 can then proceed to process the input windows to remove noise from the middle window by analyzing the middle window in the context of the surrounding windows to determine what signals in the middle window are likely to be background/environmental noise. Based on the processing, the neural network 206 can compute a vector 208 representing the auditory signal 102 with the unwanted noise signals removed.

The above processes described with respect to FIGS. 2A-2C, are in improvement over conventional systems because they apply non-causal techniques to de-noise the auditory signal 102. That is, as opposed to conventional systems, the deep learning and de-noising network 108 uses both information from the past and the future of the window to be de-noised to perform the de-noising. Typically, systems only use past or present information (i.e., are causal). The non-causal nature allows the deep learning and de-noising network 108 to better determine what frequencies are likely to correspond to background/environmental noise. This is because the deep learning and de-noising network 108 can perform the de-noising processing over a set of overlapping windows and can correlate past and future frequencies with one another to better assess which of the frequencies from the past and future are likely to be noise signals based on how they evolve over time.

### Wavelet Packet Decomposition

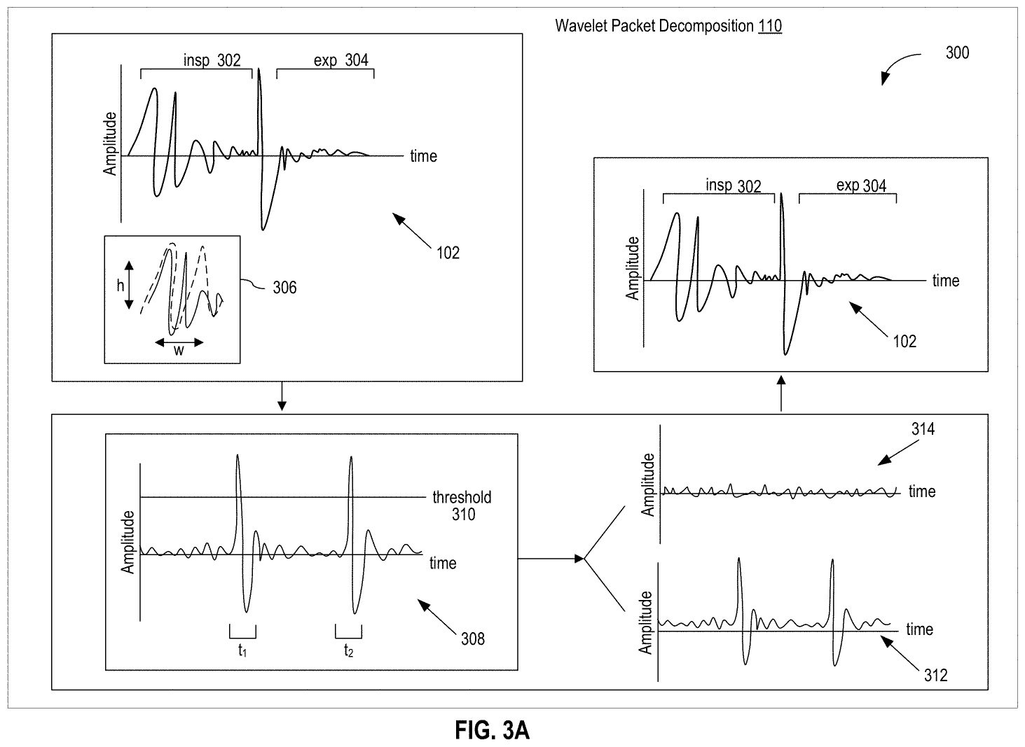

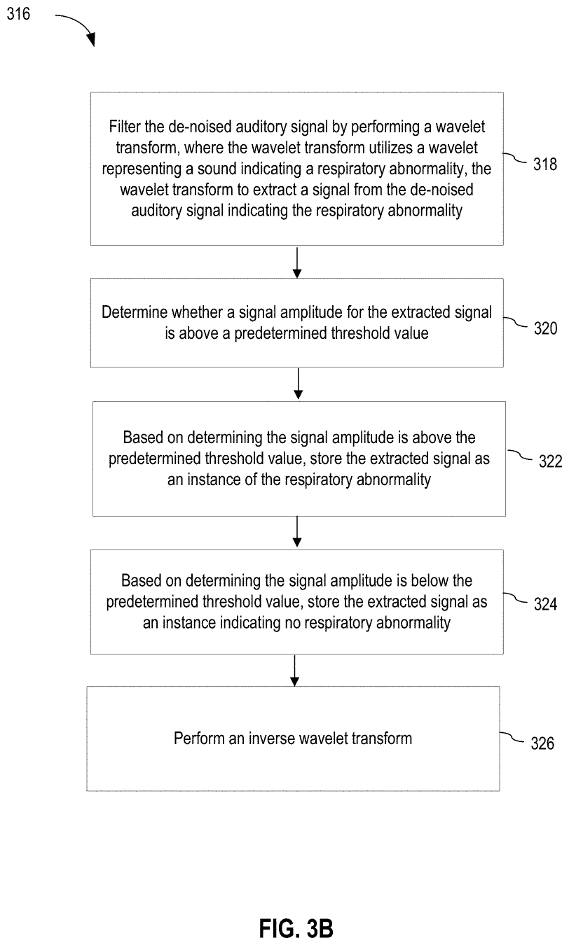

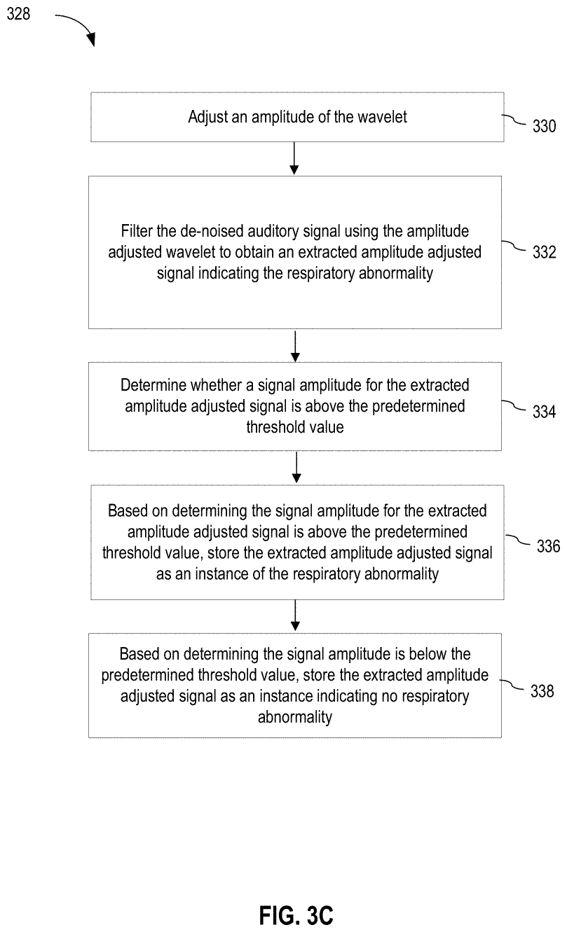

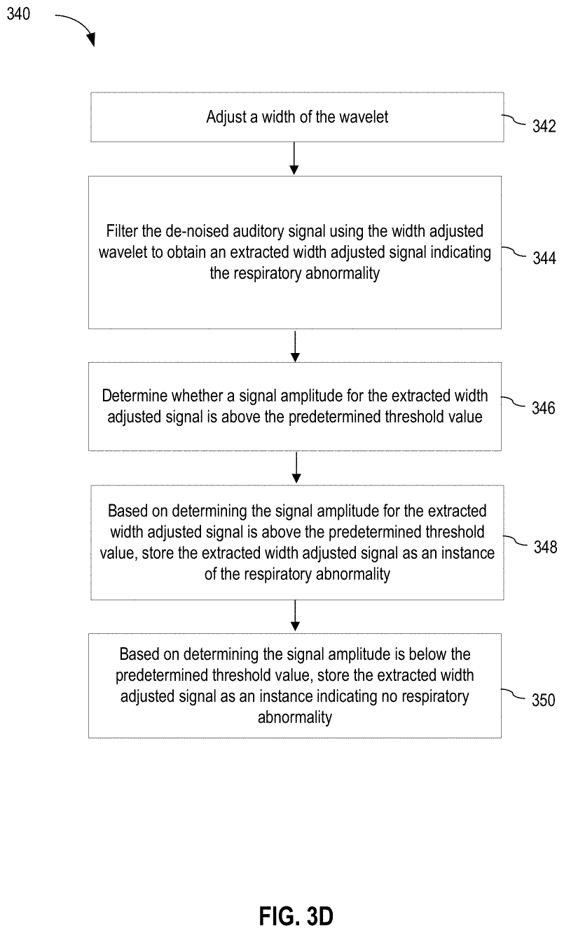

In aspects, once the auditory signal 102 is de-noised, control can be passed to a wavelet packet decomposition module 110 shown in FIG. 1. A more detailed view of the operation of the wavelet packet decomposition module 110 is shown in FIG. 3A. FIG. 3A shows a control flow 300 for how the auditory signal 102 is decomposed to its components using a wavelet packet decomposition module 110 according to aspects. The purpose of decomposing the auditory signal 102 is to extract portions of the auditory signal 102 that indicate respiratory abnormalities, such as crackles, and those portions that do not. This information can then be used to determine where and how many times in a patient's respiratory cycle the respiratory abnormality occurs.

In aspects, the decomposition process can begin by having the wavelet packet decomposition module 110 receive the auditory signal 102. For the purposes of discussion with respect to FIG. 3A, it is assumed that the auditory signal 102 is converted back or is otherwise in a time-series format, where the amplitude of the lung sounds recorded is plotted over time (i.e., time-series—as opposed to spectrogram representation where frequency of the lung sounds is plotted versus time). In aspects, the auditory signal 102 can have portions related to inhalation/inspiration 302 and an exhalation/expiration 304.

In aspects, in order to decompose the auditory signal 102 into its sub-components, the wavelet packet decomposition module 110 will apply a plurality of mother wavelets 306 to the auditory signal 102 to filter for instances of a respiratory abnormality. The mother wavelets 306 refer to archetypal signals that represent a respiratory abnormality. For example, and as shown in FIG. 3A, mother wavelets 306 are shown. These mother wavelets 306 can represent, for example, what a typical crackle sound will look like if plotted in time series. In aspects, the mother wavelets 306 can differ from each other by each having different scaling. For example, amplitudes (represented as “h” in FIG. 3A) and widths (represented as “w” in FIG. 3A) can be adjusted for each of the mother wavelets 306 so that the archetypal crackle represented by the mother wavelets 306 differs slightly. The purpose of doing this is to capture all the variations of a crackle sound from the auditory signal 102.

In aspects, the wavelet packet decomposition module 110 can then apply each of the mother wavelets 306 to the auditory signal 102 through a wavelet packet transform process. A person skilled in the art will know how a wavelet packet transform is performed, thus the details of the transform will not be discussed in detail. By performing the wavelet packet transform using the mother wavelets 306, a further signal 308 can be generated indicating all the potential crackle sounds that occur in the auditory signal 102. By way of example, FIG. 3A shows further signal 308 indicating potential crackles at t1 and t2. In aspects, once the further signal 308 is generated with all the potential crackle sounds extracted, a predetermined threshold 310 can be defined such that if the amplitudes of the potential crackle sounds are above the predetermined threshold 310, the potential crackle sounds can be stored as an instance indicating a respiratory abnormality, and if the amplitudes of the potential crackle sounds are below the predetermined threshold 310 the potential crackle sounds can be stored as an instance indicating no respiratory abnormality. In aspects, the further signal 308 can be compared to the predetermined threshold 310 to see at what points the amplitudes of the further signal 308 are above or below the predetermined threshold 310. By way of example FIG. 3A, shows the results of this comparison. Plot 312 shows stored instances of when the amplitudes are above the predetermined threshold 310 and a further plot 314 shows stored instances of when the amplitudes are below the predetermined threshold 310. In this way, the auditory signal 102 can be decomposed into its sub-components indicating when a crackle occurs within the auditory signal 102 or does not.

In aspects, once the auditory signal 102 is decomposed and the information regarding crackles is extracted, an inverse wavelet transformation process can be performed on the decomposed signals (e.g., those shown by example plots 312 and further plot 314) to reconstruct the auditory signal 102. This can be done for signals extracted and decomposed for all mother wavelets 306, which can then be reconstructed and combined to reconstruct the auditory signal 102 by using a mean value for the combined signal. The purpose of reconstructing the auditory signal 102 is so that the auditory signal 102 that was received by the wavelet packet decomposition module 110 can be used in further processes of the system 100 to determine a respiratory cycle for the patient. How this is performed will be described further below. A person skilled in the art will know how to perform an inverse wavelet transformation, thus the details of the inverse transform will not be discussed in detail. For the purposes of discussion with respect to FIG. 3A, it is assumed that the auditory signal 102 is reconstructed.

The wavelet packet transform process performed by the wavelet packet decomposition module 110, is unique in several ways. First, unlike traditional wavelet transforms, the transform used by the wavelet packet decomposition module 110 can be obtained by iterating the transform on both the detail (wavelet) and approximation (scaling) coefficients of the mother wavelets 306. Thus, for a given transformation level j of x[n], where n=1, . . . , N, the wavelet packet transform decomposes the input signal into k=1, . . . 2^{j} subbands with corresponding wavelet coefficients w′k(m), where m=1, . . . , N/2^{j}. in aspects, each of the coefficients w^{j}ₖ(m) can be scored based on equation (1) below.

$${M}_{k}^{j} ( m ) = \{ \begin{matrix}1 , & \vert {w}_{k}^{j} ( m ) \vert \geq {P}_{1} * {σ}_{k}^{j} \\ 0 , & else\end{matrix} \qquad ( 1 )$$

In equation (1), σ^{j}ₖ is the standard deviation of the wavelet coefficients in the kth subband of the level j. P₁ is a multiplicative factor. In a preferred aspect, P₁ equals three, and is determined empirically using training data. In aspects, a total score can be quantified for all k subbands of the level j using equation (2) below.

$${N}^{j} ( m ) = {\sum }_{k = 1}^{{2}^{j}} {M}_{k}^{j} ( m ) \qquad ( 2 )$$

In aspects, the predetermined threshold 310 can be defined using equation (3) below.

$${λ}^{j} = {P}_{2} * \frac{1}{L / {2}^{j}} {\sum }_{m = 1}^{L / {2}^{j}} {N}^{j} ( m ) \qquad ( 3 )$$

In equation (3), P₂ can be another multiplicative factor. In a preferred aspect, P₂ equals 2.5 and is determined empirically using training data. In aspects, the wavelet packet transform can be applied to the auditory signal 102 by partitioning the auditory signal 102 into a plurality of overlapping windows and applying each wavelet to the windows of length L. In aspects, the windows can overlap by a percentage, for example, 75%.

FIGS. 3B-3D show methods 316, 328, and 340 for decomposing an auditory signal 102 into its components using a wavelet packet decomposition process according to aspects. Methods 316, 328, and 340 can be performed using the wavelet packet decomposition module 110. FIG. 3B shows the steps of method 316. At step 318, the auditory signal 102 can be filtered using a wavelet transform, where the wavelet transform utilizes a mother wavelet representing a sound indicating a respiratory abnormality. Using the mother wavelet, the wavelet transform extracts a signal from the auditory signal 102 indicating the respiratory abnormality. At step 320, and assuming the predetermined threshold 310 is determined, a determination can be made whether a signal amplitude for the extracted signal is above the predetermined threshold 310. At step 322, based on determining the signal amplitude is above the predetermined threshold 310, the signal amplitude can be stored as an instance of the respiratory abnormality. At step 324, based on determining the signal amplitude is below the predetermined threshold 310, the signal amplitude can be stored as an instance indicating no respiratory abnormality. At step 326, once all the determinations are made for signal amplitudes, an inverse wavelet transform can be performed to reconstruct the auditory signal 102.

FIG. 3C shows the steps of method 328. Method 328 is similar to method 316 except it is performed on an amplitude adjusted mother wavelet. That is, it is performed on a mother wavelet similar to the one used in method 316 except with its amplitude adjusted. At step 330, the mother wavelet can have its amplitude adjusted. At step 332, the auditory signal 102 can be filtered using a wavelet transform, where the wavelet transform utilizes the amplitude adjusted wavelet representing a sound indicating a respiratory abnormality. Using the amplitude adjusted wavelet, the wavelet transform extracts a signal from the auditory signal 102 indicating the respiratory abnormality. At step 334, and assuming the predetermined threshold 310 is determined, a determination can be made whether a signal amplitude for the extracted signal is above the predetermined threshold 310. At step 336, based on determining the signal amplitude is above the predetermined threshold 310, the signal amplitude can be stored as an instance of the respiratory abnormality. At step 338, based on determining the signal amplitude is below the predetermined threshold 310, the signal amplitude can be stored as an instance indicating no respiratory abnormality.

FIG. 3D shows steps of method 340. Method 340 is similar to method 316 except it is performed on a width adjusted mother wavelet. That is, it is performed on a mother wavelet similar to the one used in method 316 except with its width adjusted. At step 342, the mother wavelet can have its width adjusted. At step 344, the auditory signal 102 can be filtered using a wavelet transform, where the wavelet transform utilizes the width adjusted wavelet representing a sound indicating a respiratory abnormality. Using the width adjusted wavelet, the wavelet transform extracts a signal from the auditory signal 102 indicating the respiratory abnormality. At step 346, and assuming the predetermined threshold 310 is determined, a determination can be made whether a signal amplitude for the extracted signal is above the predetermined threshold 310. At step 348, based on determining the signal amplitude is above the predetermined threshold 310, the signal amplitude can be stored as an instance of the respiratory abnormality. At step 350, based on determining the signal amplitude is below the predetermined threshold 310, the signal amplitude can be stored as an instance indicating no respiratory abnormality.

### Respiratory Cycle Extraction

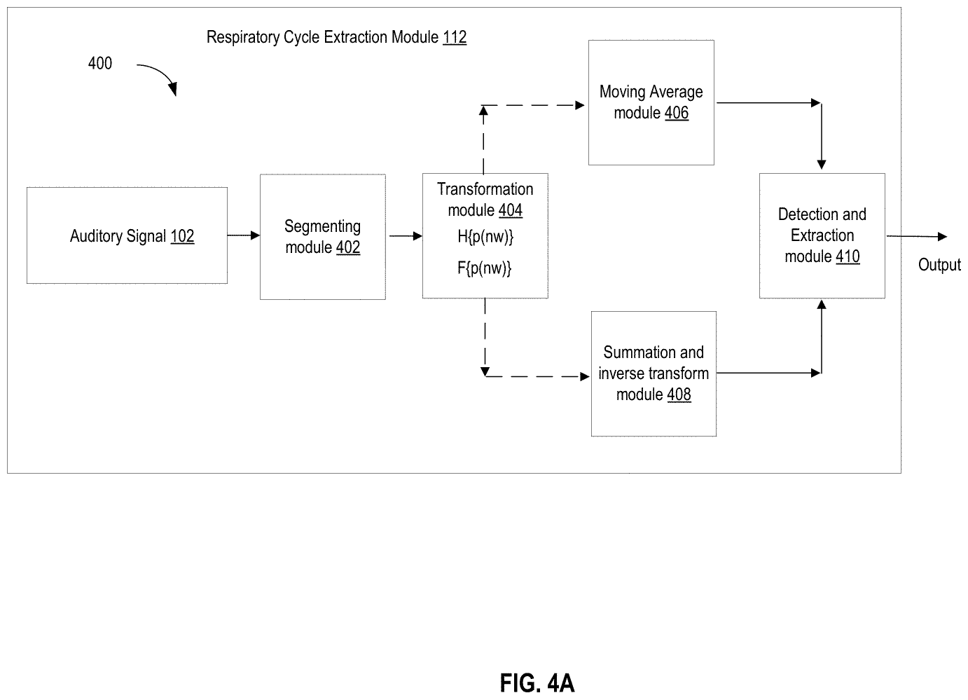

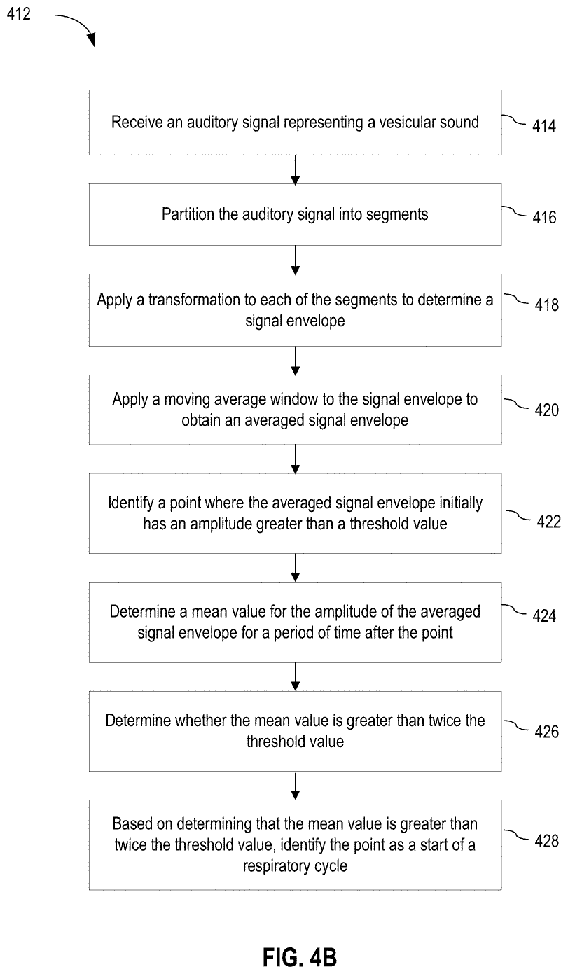

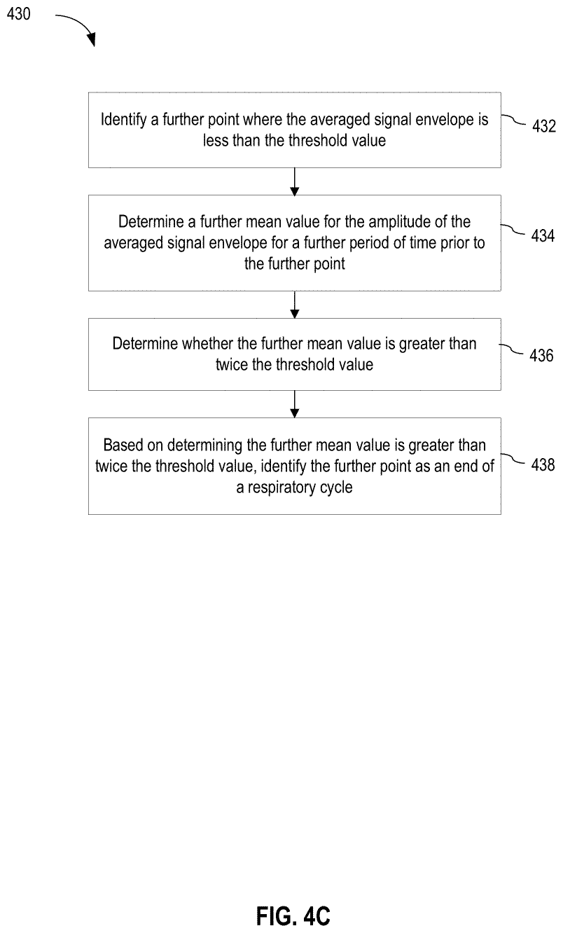

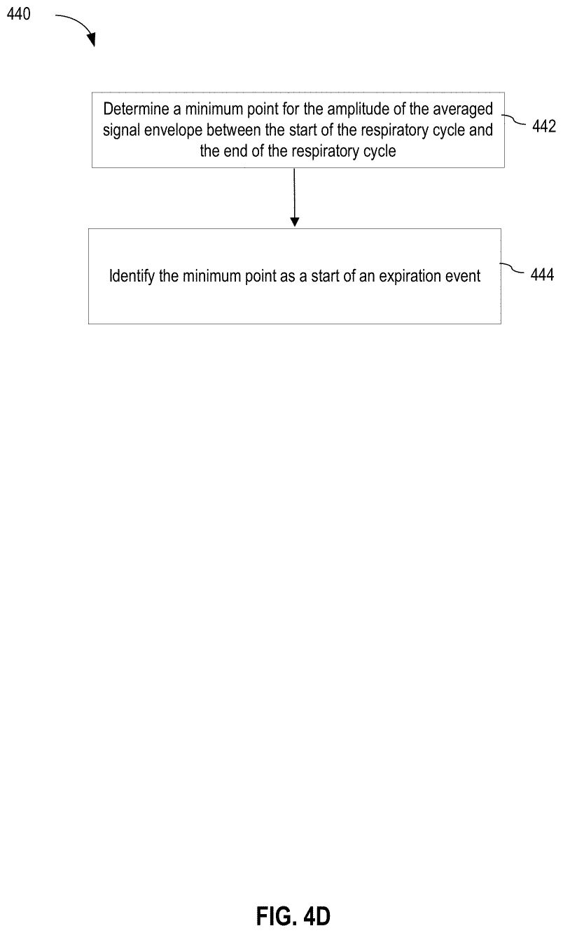

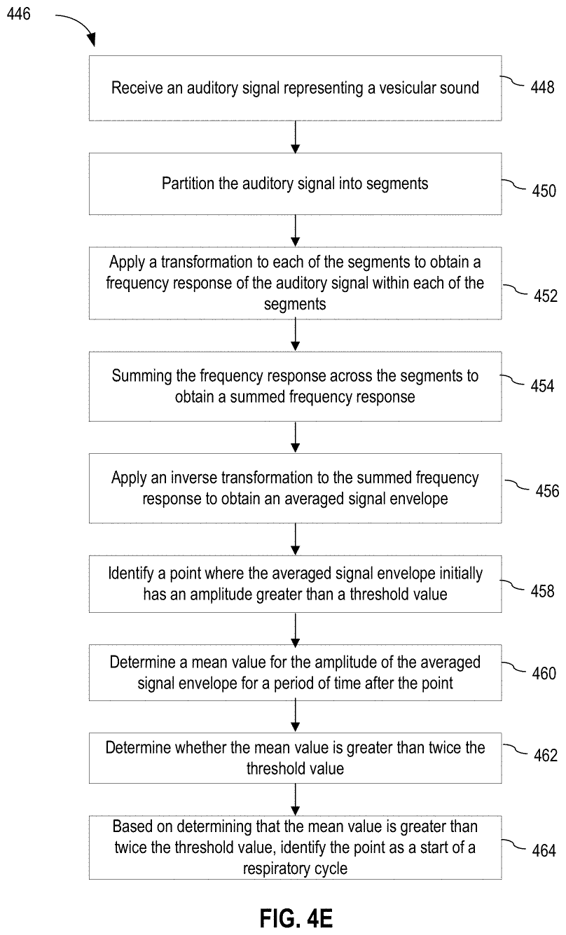

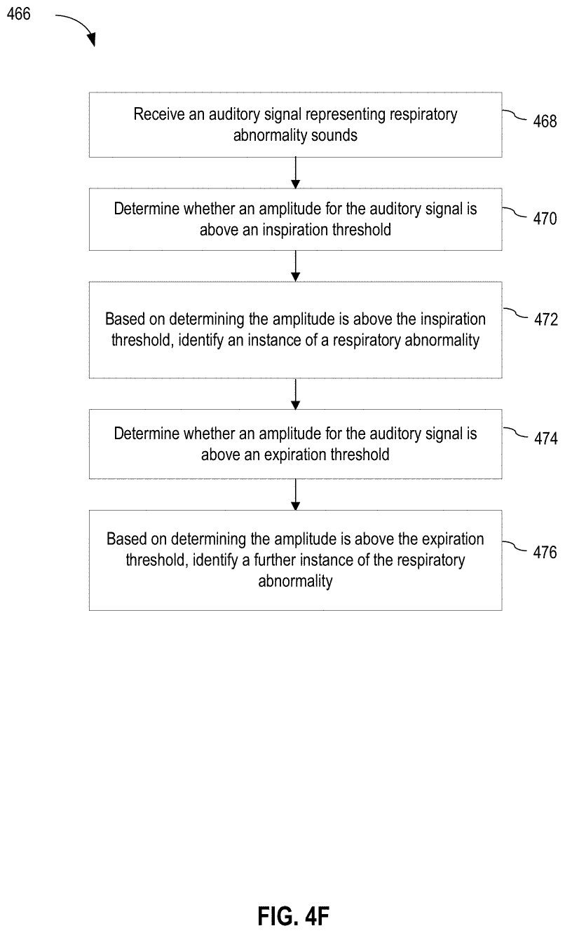

In aspects, once the wavelet packet decomposition module 110 performs its functions, control can pass to a respiratory cycle extraction module 112 shown in FIG. 1. The respiratory cycle extraction module 112 enables the extraction of a respiratory cycle based on the auditory signal 102. The auditory signal 102 can be the signal representing a vesicular sound. The auditory signal 102 can be obtained using the processes described with respect to the wavelet packet decomposition module 110 (e.g., FIGS. 3A-3D). A more detailed view of the functioning of the respiratory cycle extraction module 112 is shown in FIG. 4A.

FIG. 4A shows a control flow 400 for extracting a respiratory cycle using the respiratory cycle extraction module 112 according to aspects. In aspects, control flow 400 can begin by having the respiratory cycle extraction module 112 receive the auditory signal 102. For the purposes of discussion, it is assumed that the auditory signal 102 is in time-series format. In aspects, once received, the auditory signal 102 can be input into a segmentation module 402. The segmentation module 402 can enable the partitioning of the auditory signal 102 into segments. The segments refer to windows over a timeframe. For example, the segments can partition the auditory signal 102 into three-second windows and one-second window steps. In aspects, once partitioned, the auditory signal 102 can be transmitted to a transformation module 404. The transformation module 404 can apply a transformation to each of the segments to determine a signal envelope for each of the segments. In aspects, the transformation can be either a Hilbert transformation or a FFT.

Assuming the case where a Hilbert transformation is applied, as a result of applying the Hilbert transformation a corresponding instantaneous signal envelope of each of the windows of the auditory signal 102 can be obtained. The envelope can be calculated using equation (4) below.

pₑ(n_{w})=\sqrt{p_{H}(n_{w})²+p(n_{w})²} (4)

In equation (4), p_{H}(n_{w}) represents the Hilbert transformed envelope for each window, p(n_{w}) is a segmented window of the auditory signal 102, and pₑ(n_{w}) is the instantaneous envelope for each segmented window. In aspects, once the signal envelope is generated, control can pass to a moving average module 406. The moving average module 406 can apply a moving average window to the signal envelope to obtain an averaged signal envelope. The purpose of doing this is to smooth out the signal envelope, to have a cleaner signal that produces a more pronounced local minima between breath cycles and less prominent minima between inspiration and expiration of a single cycle. As a part of applying a moving average window, an autocorrelation of the signal envelope can be calculated using equation (5) below.

$${R}_{pp} ( l ) = {\sum }_{n = - ∞}^{∞} x ( n + l ) x ( n ) , l = 0 , \pm 1 , \pm 2 , … \qquad ( 5 )$$

In equation (5), x(n) represents the signal, and x(n+l) is a shifted/lagged version of the signal envelope. Rₚₚ(l) represents the similarity with respect to the lag. Since the respiratory signals are periodic, the autocorrelation shows significant peaks when the lag is roughly equal to a single respiratory cycle length. In aspects, equation (5) can be used to estimate the respiratory rate as the average distance between peaks in Rₚₚ(l). In aspects, the estimated respiratory rate (which can be represented as Ř) can then be used to apply a lagging moving average window from [t−α*(1/Ř), t] at sample t, with α equal to 0.5, where a is determined empirically. Based on applying the moving average window, the averaged signal envelope can be obtained.

In aspects, once the averaged signal envelope is obtained, control can pass to the detection and extraction module 410. The detection and extraction module 410 can use the averaged signal envelope to identify respiratory pauses indicative of the beginning and end of an inspiration (inhaling) or the beginning of an expiration (exhaling). For example, in aspects, the detection and extraction module 410 can identify respiratory pauses. For example, a local minimum signal pₘᵢₙ(n) can be extracted from the averaged signal envelope using a moving minimum-value window centered at n with length of β*(1/Ř), with β equal to 0.5, where β is determined empirically. In aspects, the points can be identified as respiratory pauses where the averaged signal envelope equals pₘᵢₙ(n).

In aspects, the detection and extraction module 410 can also determine a threshold value. In aspects, the threshold value can be used when detecting the beginning and end of a respiratory cycle. For example, when amplitudes of the auditory signal 102 are above the threshold value it can indicate a beginning of an inhalation or end of an inhalation, or the beginning of an expiration. How this is determined will be described further below. In aspects, the threshold value can be determined specifically for each auditory signal 102. For example, a recording-specific threshold value for a patient can be determined as the 75^{th} percentile of the amplitudes of pₘᵢₙ(n), which can be empirically set based on training data.

In aspects, in order to detect the beginning of a respiratory cycle, a point in the auditory signal 102 can be identified where the averaged signal envelope initially has an amplitude greater than the threshold value. In aspects, a mean value for the amplitude of the averaged signal envelop for a period of time after that point can be determined. In aspects, the period of time can equal 0.5 seconds. In aspects, the beginning of the respiratory cycle (i.e., beginning of inhalation/inspiration event) can be determined at the time instance n where the mean value is greater than twice the threshold value.

In aspects, the end of the respiratory cycle (i.e., end of an expiration/exhale event) can be determined based on identifying a further point where the averaged signal envelope is less than the threshold value. In aspects, a further mean value for the amplitude of the averaged signal envelope for a further period of time prior to the further point can be determined. In aspects, the further period of time can equal 0.5 seconds. In aspects, the end of the respiratory cycle can be determined as the further point where the further mean value is greater than twice the threshold value.

In aspects, the beginning of an expiration/exhale event can be determined by determining a minimum point for the amplitude of the averaged signal envelope between the start of the respiratory cycle and the end of the respiratory cycle, and identifying the minimum point as a start of the expiration/exhale event. In this way, a respiratory cycle can be extracted by applying a Hilbert transformation to the auditory signal 102.

Assuming now that the transformation applied is a FFT, the auditory signal 102 can, similar to what was described with respect to the Hilbert transformation, be partitioned into segments. In aspects, each of the segments can have a FFT applied to it to obtain a frequency response of the auditory signal 102 within each of the segments. In aspects, the frequency response across all the segments can then be summed to obtain an averaged signal envelope in the frequency domain. In aspects, in order to obtain the time-series signal for the averaged signal envelope, an inverse transformation can be applied to the summed frequency response. The inverse transformation can be an inverse FFT. Once obtained, the averaged signal envelope can be processed using the same processes described with respect to the Hilbert transformation to extract the respiratory cycle.

While, either transformation can be applied to the auditory signal to extract the respiratory cycle, the use of the FFT provides an improved method of extracting respiratory cycle because it provides a faster way to extract the respiratory cycle due to the nature of the FFT algorithm. Thus, the aforementioned processes improve computers by providing a novel and fast method that allows computers to extract respiratory cycles from a patient's lung sounds. The extracted respiratory cycle can be used, along with other extracted data that will be described further below, to determine where in the respiratory cycle respiratory abnormalities occur. By being able to do this, the methods described can provide a fully automated way to analyze a patient's lung sounds without the need for human interpretation. Moreover, the processes allow for the precise measurement of where respiratory abnormalities occur within a respiratory cycle, which cannot be done without the aid of these computer-implemented techniques. Additionally, the extraction of the respiratory cycle can be done using these methods on the fly and in real-time. Thus, doctors or care givers can obtain information about a patient's breathing cycles in real-time from when they record the patient's lung sounds to determine where and how often a respiratory abnormality happens with the patient's respiratory cycle.

FIGS. 4B-4E are exemplary methods 412, 430, 440, and 446 of extracting a respiratory cycle according to aspects. Methods 412, 430, 440, and 446 can be performed using the respiratory cycle extraction module 112. Method 412 shown in FIG. 4B shows the steps when applying a Hilbert transformation to the auditory signal 102 to extract the respiratory cycle. At step 414, an auditory signal 102 can be received. The auditory signal 102 can represent a vesicular sound. At step 416, the auditory signal 102 can be partitioned into segments. At step 418, a transformation to each of the segments can be applied to determine a signal envelope. At step 420, a moving average window can be applied to the signal envelope to obtain an averaged signal envelope. The averaged signal envelope represents a smoothed out version of the signal envelope. At step 422, a point can be identified where the averaged signal envelope initially has an amplitude greater than a threshold value. At step 424, a mean value for the amplitude of the averaged signal envelope can be determined for a period of time after the point. At step 426, a determination can be made whether the mean value is greater than twice the threshold value. At step 428, based on determining that the mean value is greater than twice the threshold value, the point can be identified as a start of the respiratory cycle.

Method 430 shown in FIG. 4C shows the steps for determining the end of a respiratory cycle. At step 432, a further point can be identified where the averaged signal envelope is less than the threshold value. At step 434, a further mean value for the amplitude of the averaged signal envelope for a further period of time prior to the further point can be determined. At step 436, a determination can be made whether the further mean value is greater than twice the threshold value. At step 438, based on determining the further mean value is greater than twice the threshold value, the further point can be identified as an end of the respiratory cycle.

Method 440 shown in FIG. 4D shows the steps for determining the beginning on an expiration event within the respiratory cycle. At step 442, a minimum point for the amplitude of the averaged signal envelope between the start of the respiratory cycle and the end of the respiratory cycle can be determined. At step 444, the minimum point can be identified as the start of an expiration event.

Method 446 shown in FIG. 4E shows the steps when applying a FFT to the auditory signal 102 to extract the respiratory cycle. At step 448, the auditory signal 102 can be received. The auditory signal 102 can represent a vesicular sound. At step 450, the auditory signal 102 can be partitioned into segments. At step 452, a transformation can be applied to each of the segments to obtain a frequency response of the auditory signal 102 within each of the segments. At step 454, the frequency response can be summed across the segments to obtain a summed frequency response. At step 456, an inverse transformation can be applied to the summed frequency response to obtain an averaged signal envelope. The inverse transformation can be an inverse FFT. At step 458, a point can be identified where the averaged signal envelope initially has an amplitude greater than a threshold value. At step 460, a mean value for the amplitude of the averaged signal envelope for a period of time after the point can be determined. At step 462, a determination can be made whether the mean value is greater than twice the threshold value. At step 464, based on based on determining that the mean value is greater than twice the threshold value, the point can be identified as the start of the respiratory cycle.

FIG. 4F shows an exemplary method 466 for counting respiratory abnormalities according to aspects. In aspects, method 466 can be performed by a crackle peak detection module 114 shown in FIG. 1. In aspects, based on the processing done by the wavelet packet decomposition module 110, as was previously described, the auditory signal 102 can be decomposed into its components indicating instances within the respiratory cycle where crackles occur. In aspects, the crackle peak detection module 114 can take the decomposed signals that are extracted, and perform the analysis needed to determine how many crackles occur within the respiratory cycle. At step 468 of method 466, the crackle peak detection module 114 can receive an auditory signal 102 representing respiratory abnormality sounds. The auditory signal 102 can represent a signal where only the respiratory abnormalities are present. For example, it can be a signal showing only the crackles occurring within the respiratory cycle. At step 470, a determination can be made whether an amplitude for the auditory signal is above an inspiration threshold. The inspiration threshold can represent a threshold value that can be predetermined empirically and represent a threshold value above which a sound can be classified as a crackle. At step 472, based on determining the amplitude is above the inspiration threshold, an instance of the respiratory abnormality can be identified. At step 474, a determination can be made whether an amplitude for the auditory signal is above an expiration threshold. The expiration threshold, similar to the inspiration threshold can represent a threshold value that can be predetermined empirically and represent a threshold value above which a sound can be classified as a crackle. The expiration threshold can relate to the period of time within the respiratory cycle when the patient is exhaling. Because the signal during exhaling has a different power distribution than during inhaling, and the amplitudes of the signal during exhaling are smaller, the expiration threshold can be specifically defined such to determine threshold values above which crackles are determined. This is necessary because if only the inspiration threshold is used, signals that might be crackles might not be captured because the threshold value would be too high. In aspects, the thresholds described can be customized for each patient and set based on an average power of the auditory signal 102 for the patient. In aspects, and as shown in step 476, based on determining the amplitude is above the expiration threshold, a further instance of the respiratory abnormality can be identified.

### Hardware Components

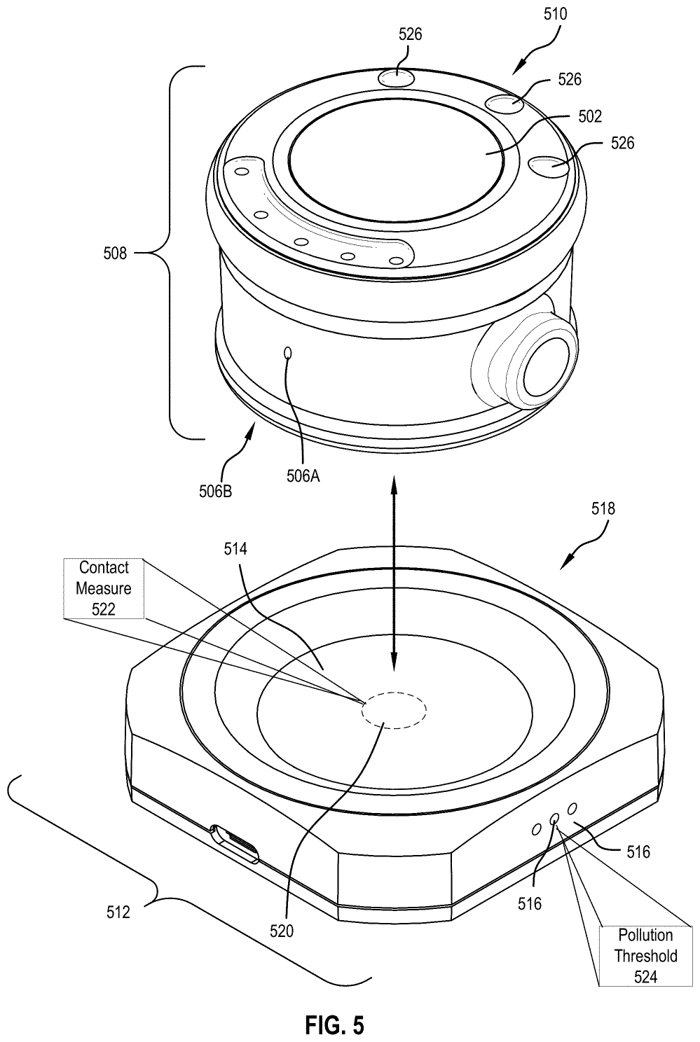

As indicated with respect to FIG. 1, the system 100 described above can have its operations performed on hardware components such as a digital stethoscope. The following is a description of a digital stethoscope 510 that can be used to implement the functions of system 100. FIG. 5 shows an example digital stethoscope 510 and base station 518 that can be used to implement the functions of the system 100 according to aspects.

The digital stethoscope 510 is an acoustic device for detecting and analyzing noises from a patient's body. The patient can be, for example, a human or an animal. The noises, from the patient's body can be for example a cough, a wheeze, a crackle, a breathing pattern, a heartbeat, a chest motion representing a patient's respiratory cycle, or a combination thereof.

The digital stethoscope 510 can include one or more components. For example, in aspects, the digital stethoscope 510 can include a display unit 502, one or more microphones 506, and a first housing 508. The display unit 502 can be any graphical user interface such as a display, a projector, a video screen, a touch screen, or any combination thereof that can present information detected or generated by the digital stethoscope 510 for visualization by a user of the system 100. The display unit 502 can enable the visual presentation of information detected or generated by the digital stethoscope 510.

For example, in aspects, the display unit 502 can enable the visual presentation of the noises detected, by for example, displaying a plot of the sound frequencies detected over time, displaying a decibel level of the sounds detected, or displaying a value or visual indicator representing the classification of the noises generated, for example “normal” or “abnormal,” or display the number of respiratory abnormalities counted within a respiratory cycle. In aspects, if the digital stethoscope 510 classifies a noise as being “abnormal,” the display unit 502 can display an indicator, such as a red colored light, or a message indicating that the noise is “abnormal.” Alternatively, if the digital stethoscope 510 classifies the noise as being “normal,” the display unit 502 can display an indicator, such as a green colored light, or a message indicating that the noise is “normal.”

The display unit 502 can further present other information generated by the digital stethoscope 510, such as a power level indicator indicating how much power the digital stethoscope has, a volume indicator indicating the volume level of output noises being output by the digital stethoscope 510, or a network connectivity indicator indicating whether the digital stethoscope 510 is connected to a device or computer network such as a wireless communication network or wired communication network. The aforementioned information are merely exemplary of the types of information that the display unit 502 can display, and are not meant to be limiting.

In aspects, the display unit 502 can further include one or more buttons 526 that can be used by the user of the system 100 to enable interaction with the digital stethoscope 510. For example, the buttons 526 can provide functionality such as powering the digital stethoscope 510 on or off or enable the digital stethoscope 510 to start or stop recording the noises.

In aspects, the digital stethoscope 510 can further include one or more microphones 506A and B. The microphones 506A and B enable the digital stethoscope 510 to detect and convert the noises into electrical signals for processing by the digital stethoscope 510, or a further device such as the base station 518. Microphone 506A is mounted on a perimeter side of stethoscope 110 to detect noises external to the patient's body. The noises originating from external to the patient's body can be for example background noise, white noise, or a combination thereof. Microphone 506B may be mounted on a side reverse of display 102 and may detect noises originating from the patient's body.

The microphones 506A and B can be standalone devices or can be arranged in an array configuration, where the microphones 506 operate in tandem to detect the noises. In aspects, each microphone in the array configuration can serve a different purpose. For example, each microphone in the array configuration can be configured to detect and convert into electrical signals the noises at different frequencies or within different frequency ranges such that each of the microphones 506 can be configured to detect specific noises. The noises detected by the microphones 506 can be used to generate the values for classifying the noises as “normal” or “abnormal,” and can be further used to predict the respiratory event or respiratory condition in the future.

The digital stethoscope 510 can further have a first housing 508 enclosing the components of the digital stethoscope 510. The first housing 508 can separate components of the digital stethoscope 510 contained within from other components external to the first housing 508. For example, the first housing 508 can be a case, a chassis, a box, or a console. In aspects, for example, the components of the digital stethoscope 510 can be contained within the first housing 508. In other aspects, some components of the digital stethoscope 510 can be contained within the first housing 508 while other components, such as the display unit 502, the microphones 506, the buttons 526, or a combination thereof, can be accessible external to the first housing 508. The aforementioned are merely examples of components that can be contained in or on the first housing 508 and are not meant to be limiting. Further discussion of other components of the digital stethoscope 510 will be discussed below.

A base station 518 can also be included to be used in conjunction with the digital stethoscope 510. The base station 518 is a special purpose computing device that enables computation and analysis of the noises obtained by the digital stethoscope 510 in order to detect the respiratory abnormality, or to predict the respiratory event or respiratory condition in the future. The base station 518 can provide additional or higher performance processing power compared to the digital stethoscope 510. In aspects, the base station 518 can work in conjunction with the digital stethoscope 510 to detect, amplify, adjust, and analyze noises from a patient's body by, for example, providing further processing, storage, or communication capabilities to the digital stethoscope 510. In other aspects, the base station 518 can work as a standalone device to detect, amplify, adjust, and analyze noises to detect the respiratory abnormality, or to predict the respiratory event or respiratory condition in the future.

The base station 518 can analyze the noises captured by digital stethoscope 510. For example, in aspects, the base station 518 can generate values classifying the noises detected as “normal” or “abnormal.” The collection, filtering, comparison, and classification of the noises by the base station 518 will be discussed further below.

The base station 518 can include one or more components. For example, in aspects, the base station 518 can include a charging pad 514, one or more air quality sensors 516, a contact sensor 520, and a second housing 512. The charging pad 514 can enable the electric charging of the digital stethoscope 510, through inductive charging where an electromagnetic field is used to transfer energy between the charging pad 514 and a further device, such as the digital stethoscope 510, using electromagnetic induction.

In aspects, the charging pad 514 can enable electric charging of the digital stethoscope 510 upon detecting contact or coupling, via the contact sensor 520, between the digital stethoscope 510 and the charging pad 514. For example, in aspects, if the digital stethoscope 510 is coupled to the charging pad 514 by physical placement of the digital stethoscope 510 on the charging pad 514, the contact sensor 520 can detect a weight or an electromagnetic signal produced by the digital stethoscope 510 on the charging pad 514, and upon sensing the weight or the electromagnetic signal, enable the induction process to transfer energy between the charging pad 514 and the digital stethoscope 510.

In other aspects, if the digital stethoscope 510 is coupled to the charging pad 514 by placing the digital stethoscope 510 in proximity of the charging pad 514 without physically placing the digital stethoscope 510 on the charging pad 514, the contact sensor 520 can detect an electric current or a magnetic field from one or more components of the digital stethoscope 510 and enable the induction process to transfer energy between the charging pad 514 and the digital stethoscope 510.

The contact sensor 520 is a device that senses mechanical or electromagnetic contact and gives out signals when it does so. The contact sensor 520 can be, for example, a pressure sensor, a force sensor, strain gauges, piezoresistive/piezoelectric sensors, capacitive sensors, elastoresistive sensors, torque sensors, linear force sensors, an inductor, other tactile sensors, or a combination thereof configured to measure a characteristic associated with contact or coupling between the digital stethoscope 510 and the charging pad 514. Accordingly, the contact sensor 520 can output a contact measure 522 that represents a quantified measure, for example, a measured force, a pressure, an electromagnetic force, or a combination thereof corresponding to the coupling between the digital stethoscope 510 and the charging pad 514. For example, the contact measure 522 can detect one or more force or pressure readings associated with forces applied by the digital stethoscope 510 on the charging pad 514. The contact measure 522 can further detect one or more electric current or magnetic field readings associated with placing the digital stethoscope 510 in proximity of the charging pad 514.

In aspects, the base station 518 can further include one or more air quality sensors 516. The air quality sensors 516 are devices that detect and monitor the presence of air pollution in a surrounding area. Air pollution refers to the presence of or introduction into the air of a substance which has harmful or poisonous effects on the patient's body. For example, the air quality sensors 516 can detect the presence of particulate matter or gases such as ozone, carbon monoxide, sulfur dioxide, nitrous oxide, or a combination thereof that can be poisonous to the patient's body, and in particular poisonous to the patient's respiratory system.

In aspects, based on the air quality sensors 516 detecting the presence of air pollution, the base station 518 can determine whether the amount of air pollution poses a health risk to the patient by, for example, comparing the levels of air pollution to a pollution threshold 524 to determine whether the levels of air pollution in the surrounding area of the base station 518 pose a health risk to the patient. The pollution threshold 524 refers to a pre-determined level for particulate matter or gases measured in micrograms per cubic meter (μg/m³), parts per million (ppm), or parts per billion (ppb), that if exceeded poses a health risk to the patient

For example, in aspects, if the air quality sensors 516 detect the presence of sulfur dioxide above 75 ppb in the air surrounding the base station 518, the base station 518 can determine that the air pollution in the surrounding area poses a health risk to the patient. The detection of air pollution can further be used for detecting the respiratory abnormality or to predict the respiratory event or respiratory condition in the future and in the patient by allowing the system 100 to determine what factors are contributing to the “normal” or “abnormal” classification of the noises, or what factors are contributing to the data detected and generated by the system 100 which can be used to predict a respiratory event or respiratory condition in the future.

The base station 518 can further have a second housing 512 enclosing the components of the base station 518. The second housing 512 can separate components of the base station 518 contained within, from other components external to the second housing 512. For example, the second housing 512 can be a case, a chassis, a box, or a console. In aspects, for example, the components of the base station 518 can be contained within the second housing 512. In other aspects, some components of the base station 518 can be contained within the second housing 512 while other components, such as the charging pad 514 or the air quality sensors 516 can be accessible external to the second housing 512. The aforementioned are merely examples of components that can be contained in or on the second housing 512 and are not meant to be limiting. Further discussion of other components of the base station 518 will be discussed below.

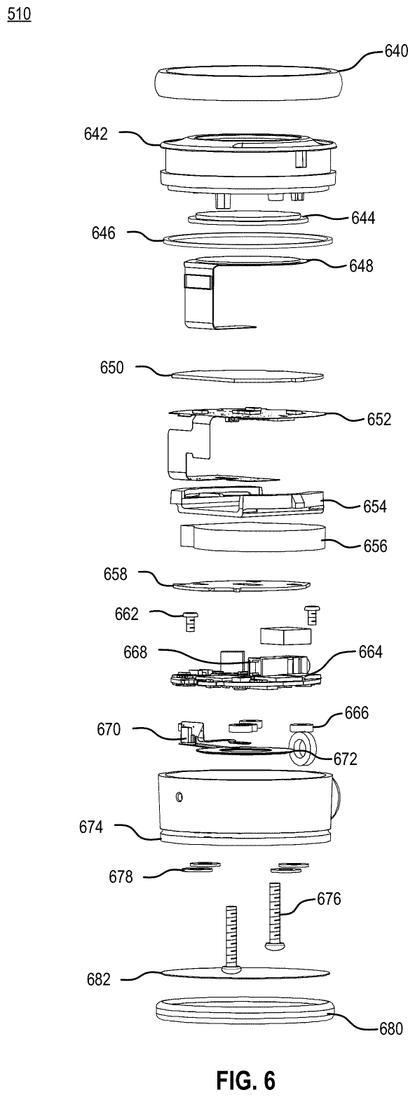

FIG. 6 shows an exemplary architecture of the digital stethoscope 510 according to aspects. In aspects, the digital stethoscope 510 can include, alternatively or additionally:
a grip ring 640 located around a first upper portion 642 of the first housing 508 which provides a gripping surface for a user of the digital stethoscope 510 to hold the digital stethoscope 510;
a glass lens 644 of the display unit 502, which protects the display components, such as for example liquid crystal displays (LCD) of the display unit 502. The glass lens 644 can sit on top of a housing gasket 646, which stabilizes and holds the glass lens 644; a display housing unit 648, on which the housing gasket 646 sits and which contains the components of the display unit 502, such as for example the LCDs;
a flex backing 650 which on which the display housing 648 sits and which provides stability for the display housing 648;
a flex assembly 652, on which the flex backing 650 sits and which provides stability for the flex backing 650;
a retainer clip 654 which holds the flex assembly 652 in place;
a battery housing 656, to which a battery board 658 can couple, and which can hold battery components of the digital stethoscope 510;
a first printed circuit board assembly 664, which can hold the circuitry, including any processors, memory components, active and passive components, or a combination thereof, of the digital stethoscope 510;
one or more first screws 662 that couples the first printed circuit board assembly 664 to the other components of the digital stethoscope 510;
an audio jack 668 to allow output of noise signals detected by the digital stethoscope 510;
a microphone assembly 670, on which the microphones 506 can be housed;
components such as an O-ring 672 and one or more coils 666 that couple the microphone assembly 670 to the first printed circuit board assembly 664.
a first bottom portion 674 of the first housing 508 on which the microphone assembly 670 sits;
a diaphragm membrane 682 which forms the bottom surface of the digital stethoscope 510, and which is coupled to the first bottom portion 674 of the first housing 508 with one or more second screws 676 and one or more washers 678; and
a diaphragm ring 680 coupled to the diaphragm membrane 682, which provides a gripping surface for the first bottom portion 674 of the digital stethoscope 510, such that the digital stethoscope 510 does not slip when placed on a surface.

The aforementioned components are merely exemplary and represent an aspect of the digital stethoscope 510.

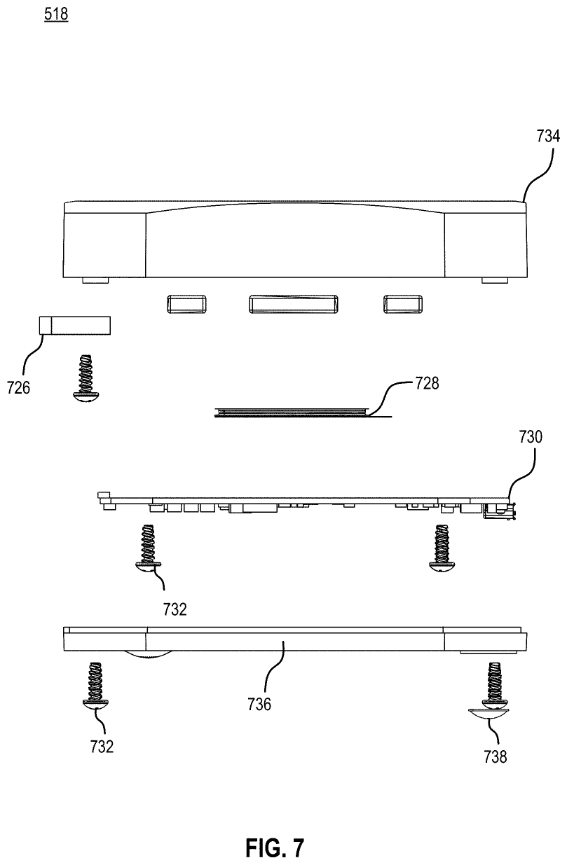

FIG. 7 shows an exemplary architecture of the base station 518 according to aspects. In aspects, the base station 518 can include, alternatively or additionally: a second upper portion 734 of the second housing 512;
a second printed circuit board assembly 730 which can hold the circuitry, including any processors, memory components, active and passive components, or a combination thereof, of the base station 518;
one or more third screws 732 that couples the second printed circuit board assembly 730 to the second upper portion 734 of the second housing 512 via one or more second connectors 726;
one or more coils 728, coupled to the second printed circuit board assembly 730, which can detect the weight or the electromagnetic signal produced by the digital stethoscope 510 on the base station 518;
a second bottom portion 736 of the second housing 512, which forms the bottom surface of the base station 518; and one or more bumpers 738 to cover and protect the third screws 732.

The aforementioned components are merely exemplary and represent an aspect of the base station 518.

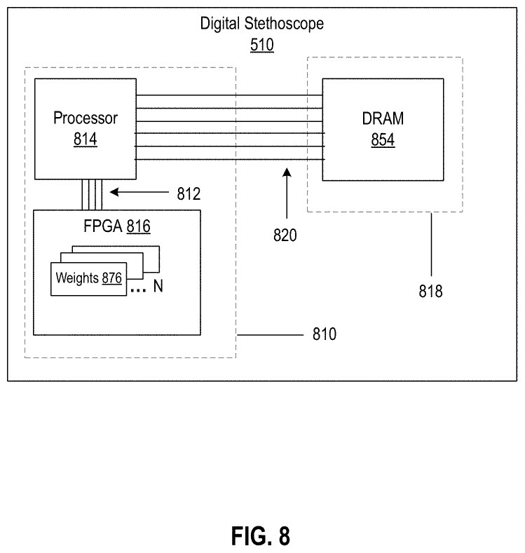

FIG. 8, shows exemplary components of the digital stethoscope 510 according to aspects. FIG. 8 shows an aspect that includes a control unit 810 and a storage unit 818. The control unit 810 can include a processor 814 and a FPGA 816. The storage unit 818 can include a DRAM 854. The processor 814 and the FPGA 816 can be coupled using a control interface 812, which can include bus lines to transfer data. The storage unit 818 can be coupled to the control unit 810 using a storage interface 820, which can include bus bars to transfer data. In aspects, the DRAM 854 can be coupled, via the storage interface 820, to the processor 814.

In aspects, the processor 814, the FPGA 816, and the DRAM 854 can work in conjunction to process auditory signals detected by the microphones 506. In aspects, the processor 814 can act as a controller and control the coordination, communications, scheduling, and transfers of data between the FPGA 816, the DRAM 854, or other components of the digital stethoscope 510. For example, in aspects, the processor 814 can receive the auditory signal 102 from the microphones 506, and transfer the auditory signal 102 to the FPGA 816 for further processing. In aspects, once the FPGA 816 has completed its operations, the FPGA 816 can transfer the output or data generated as a result of its operations back to the processor 814, which can further transfer the output or data to the DRAM 854 for storage.

In aspects, the FPGA 816 can perform the processing of the auditory signal 102. The FPGA 816 can include one or more logic blocks, including one or more reconfigurable logic gates, that can be pre-programmed or configured to perform calculations or computations on the auditory signal 102, and to generate output or data to detect the respiratory abnormality, or to predict a respiratory event or respiratory condition in the future. The FPGA 816 can, for example, have its logic blocks preconfigured with threshold values, stored values, acoustic models, machine learned trained data, machine learning processes, configuration data, or a combination thereof that can be used to perform the processing on the auditory signal 102, the result of which is to detect the respiratory abnormality, to predict the respiratory event or respiratory condition in the future, or otherwise to perform the functions described with respect to the system 100.

For example, in aspects the FPGA 816 can be preconfigured with a machine learning models, for example a convolutional neural network model, which can have one or more weights 876 associated therewith. The weights 876 refer to values, parameters, thresholds, or a combination thereof that act as filters in the machine learning process and represent particular features of the sounds, noises, and acoustic tones of a respiratory abnormality, respiratory event, respiratory condition, or a combination thereof. The weights 876 can be iteratively adjusted based on training data.

Continuing with the example, the FPGA 816 can, in aspects, use the machine learning models, including the weights 876 to detect whether auditory signals contain a sound, noise, or acoustic tone indicative of a respiratory abnormality, or whether auditory signals are indicative of a respiratory event or respiratory condition in the future, or to perform the operations with respect to system 100 and FIGS. 1-4F.

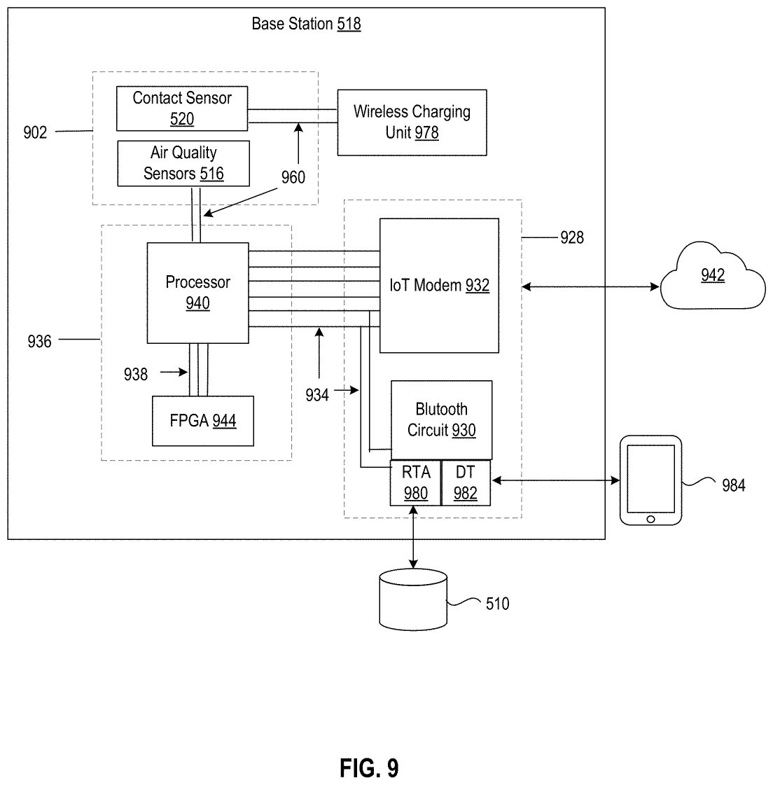

FIG. 9, shows exemplary components of the base station 518 according to aspects. FIG. 9 shows an aspect where the base station 518 includes a control unit 936, a sensor unit 902, a communication unit 928, and a wireless charging unit 978. The control unit 936 can include a processor 940 and a FPGA 944. The sensor unit 902 can include the contact sensor 520 and the air quality sensors 516. The communication unit 928 can include an IoT modem 932 and a Bluetooth circuit 930. The Bluetooth circuit 930 can further include a real time audio circuit 980 and a data transfer circuit 982. The real time audio circuit 980 and the data transfer circuit 982 can enable the base station 518 to connect to multiple devices simultaneously over a Bluetooth connection. For example, in aspects, the real time audio circuit 980 can enable a Bluetooth connection to the digital stethoscope 510 to send or receive the auditory signal 102 or a sound file containing the auditory signal 102, and the data transfer circuit 982 can enable simultaneous Bluetooth connection to a further device, such as a mobile phone 984 to communicate outputs or data generated by the base station 518 as a result of processing the auditory signal 102. In aspects, the IoT modem 932 can further be used to communicate outputs or data generated by the base station 518 to a further device, for example a remote server 942. In aspects, the IoT modem 932 can further be used to receive configuration data, such as software updates, including updated acoustic models, machine learned trained data, machine learning processes, firmware, or a combination thereof from the remote server 942. In aspects, the base station 518 can further communicate the software updates to the digital stethoscope 510 using the Bluetooth circuit 930.

The processor 940 and the FPGA 944 can be coupled using a control interface 938, which can include a bus for data transfers. The communication unit 928 can couple to the control unit 936 using a communication interface 934, which can include a bus for data transfers. The sensor unit 902 can couple to the control unit 936 using a sensor unit interface 960, which can include a bus for data transfers. The sensor unit 902 can couple to the wireless charging unit 978 using the sensor unit interface 960.

In aspects, the processor 940 can act as a controller and control the coordination, communications, scheduling, and transfers of data between the FPGA 944 and other components of the base station 518. For example, in aspects, the processor 940 can receive the auditory signal 102 from the digital stethoscope 510 via the communication unit 928, and transfer the auditory signal 102 to the FPGA 944 for further processing. In aspects, once the FPGA 944 has completed its operations, the FPGA 944 can transfer the output or data generated as a result of its operations back to the processor 940, which can further transfer the output or data to other components of the base station 518. For example, the processor 940 can further transfer the output or data to the communication unit 928 for transfer to the remote server 942, the mobile device 984, the digital stethoscope 510, or a combination thereof. The mobile device 984 can be a device associated with a user of the system 100 that the base station 518 can use to communicate the output or data generated by the base station 518, the digital stethoscope 510, the remote server 942, or a combination thereof to a user of the system 100. The mobile device 984 can be, for example, a mobile phone, a smart phone, a tablet, a laptop computer, or a combination thereof.

In aspects, the FPGA 944 can perform the processing of the auditory signal 102. The FPGA 944 can include one or more logic blocks, including one or more reconfigurable logic gates, that can be pre-programmed or configured to perform calculations or computations on the auditory signal 102, and to generate output or data generated to detect the respiratory abnormality, or to predict a respiratory event or respiratory condition in the future. The FPGA 944 can, for example, have its logic blocks preconfigured with threshold values, stored values, acoustic models, machine learned trained data, machine learning processes, configuration data, or a combination thereof that can be used to perform the processing on the auditory signal 102, the result of which is to detect the respiratory abnormality, or to predict the respiratory event or respiratory condition in the future, or otherwise perform the functions of FIGS. 1-4F.

For example, in aspects the FPGA 944 can be preconfigured with a machine learning model, for example a convolutional neural network model, which can have one or more weights 876 as shown in FIG. 8, associated therewith. In other aspects, the FPGA 944 can be preconfigured with a machine learning model, for example a long short term memory (LSTM) network model, which can have one or more weights 876 associated therewith. In aspects, the FPGA 944 can work with the remote server 942 to implement the machine learning models, for example the neural network model, or the LSTM network model, wherein the FPGA 944 and the remote server 942 can divide the processing needed to perform the computations done by the machine learning model.

Continuing with the example, the FPGA 944 can, in aspects, use the machine learning model to detect whether the auditory signal 102 contains a sound, noise, or acoustic tone indicative of a respiratory abnormality. In other aspects, the FPGA 944 can use the machine learning model to predict a respiratory event or respiratory condition in the future using auditory signals, or otherwise perform the functions of the system as described with respect to FIGS. 1-4F.

The wireless charging unit 978 can enable the electric charging of the digital stethoscope 510, through inductive charging by, for example, generating the electromagnetic field used to transfer energy between the charging pad 514 of FIG. 5, and a further device, such as the digital stethoscope 510 using electromagnetic induction. The wireless charging unit 978 can include the processors, active and passive components, circuitry, control logic, or a combination thereof to enable the inductive charging. In aspects, the wireless charging unit 978 can couple to the contact sensor 520 to enable the inductive charging. For example, in aspects, if the contact sensor 520 detects contact or coupling between the digital stethoscope 510 and the charging pad 514, the contact sensor 520 can generate the contact measure 522 of FIG. 5, which can be sent to the wireless charging unit 978. The wireless charging unit 978, upon receiving the contact measure 522 can determine that a coupling between the digital stethoscope 510 and the charging pad 514 has occurred and can activate the base station's 518 processors, active and passive components, circuitry, control logic, or a combination thereof to generate the electromagnetic field and begin transferring energy between the charging pad 514 and the digital stethoscope 510. In aspects, the wireless charging unit 978 can further power off the base station 518 during the time period in which it is charging the digital stethoscope 510 by, for example, generating a signal to the processor 940 that charging is taking place and that the components of the base station 518 should be in an off or idle mode during the time period.

In aspects, the wireless charging unit 978 can further enable the activation of the base station 518 based on determining a termination of the coupling between the digital stethoscope 510 and the charging pad 514. For example, in aspects, the wireless charging unit 978 can detect a termination of the coupling between the digital stethoscope 510 and the charging pad 514 based on a change in the contact measure 522. For example, in aspects, if the digital stethoscope 510 is removed from the charging pad 514, the contact sensor 520 can generate a contact measure 522 indicating the removal, and can send the contact measure 522 to the wireless charging unit 978. The wireless charging unit 978 upon receiving the contact measure 522 can determine that the coupling between the digital stethoscope 510 and the charging pad 514 is no longer present and can send a signal to the processor 940 to activate or power up the components of the base station 518, so that the base station 518 can perform computations and processing on auditory signals, or communicate with further devices such as the digital stethoscope 510, the mobile device 984, the remote server 942, or a combination thereof.

The above aspects are described in sufficient detail to enable those skilled in the art to make and use the disclosure. It is to be understood that other aspects are evident based on the present disclosure, and that system, process, or mechanical changes may be made without departing from the scope of an aspect of the present disclosure.

In the above description, numerous specific details are given to provide a thorough understanding of the disclosure. However, it will be apparent that the disclosure may be practiced without these specific details. To avoid obscuring an aspect of the present disclosure, some well-known circuits, system configurations, and process steps are not disclosed in detail.

The term “module” or “unit” referred to herein can include software, hardware, or a combination thereof in an aspect of the present disclosure in accordance with the context in which the term is used. For example, the software can be machine code, firmware, embedded code, or application software. Also for example, the hardware can be circuitry, a processor, a microprocessor, a microcontroller, a special purpose computer, an integrated circuit, integrated circuit cores, a pressure sensor, an inertial sensor, a microelectromechanical system (MEMS), passive devices, or a combination thereof. Further, if a module or unit is written in the system or apparatus claims section below, the module or unit is deemed to include hardware circuitry for the purpose and the scope of the system or apparatus claims.

The modules and units in the following description of the aspects can be coupled to one another as described or as shown. The coupling can be direct or indirect, without or with intervening items between coupled modules or units. The coupling can be by physical contact or by communication between modules or units.

The above detailed description and aspects of the disclosed system 100 are not intended to be exhaustive or to limit the disclosed system 100 to the precise form disclosed above. While specific examples for the system 100 are described above for illustrative purposes, various equivalent modifications are possible within the scope of the disclosed system 100, as those skilled in the relevant art will recognize. For example, while processes and methods are presented in a given order, alternative implementations may perform routines having steps, or employ systems having processes or methods, in a different order, and some processes or methods may be deleted, moved, added, subdivided, combined, or modified to provide alternative or sub-combinations. Each of these processes or methods may be implemented in a variety of different ways. Also, while processes or methods are at times shown as being performed in series, these processes or blocks may instead be performed or implemented in parallel, or may be performed at different times.

The resulting method, process, apparatus, device, product, and system is cost-effective, highly versatile, and accurate, and can be implemented by adapting components for ready, efficient, and economical manufacturing, application, and utilization. Another important aspect of the present disclosure is that it valuably supports and services the historical trend of reducing costs, simplifying systems, and increasing performance.

These and other valuable aspects of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

What is claimed is:

1. A computer-implemented method for de-noising an auditory signal, the method comprising:
partitioning, by one or more computing devices, an auditory spectrogram representing the auditory signal into a plurality of windows of equal length timeframes, wherein each of the windows indicates a frequency response of the auditory signal within each of the timeframes; and
processing, by the one or more computing devices, each of the windows using a neural network trained to remove unwanted noise signals from the auditory signal, wherein the processing comprises:
(i) identifying an odd number of consecutive windows,
(ii) identifying a middle window from the odd number of consecutive windows, wherein the middle window is a window to have the unwanted noise signals removed,
(iii) identifying an even number of windows preceding the middle window,
(iv) identifying an even number of windows following the middle window,
(v) inputting the middle window, the even number of windows preceding the middle window, and the even number of windows following the middle window into the neural network, and
(vi) computing, using the neural network, a vector representing the auditory signal with the unwanted noise signals removed.

2. The method of claim 1, further comprising repeating (i)-(vi) for each of the plurality of windows of the auditory spectrogram.

3. The method of claim 1, further comprising:
filtering, by the one or more computing devices, the auditory signal by performing a wavelet transform, wherein the wavelet transform utilizes a wavelet representing a sound indicating a respiratory abnormality, the wavelet transform to extract a signal from the auditory signal indicating the respiratory abnormality;
determining, by the one or more computing devices, whether a signal amplitude for the extracted signal is above a predetermined threshold value;
based on determining the signal amplitude is above the predetermined threshold value, storing, by the one or more computing devices, the extracted signal as an instance of the respiratory abnormality; and
based on determining the signal amplitude is below the predetermined threshold value, storing, by the one or more computing devices, the extracted signal as an instance indicating no respiratory abnormality.

4. The method of claim 3, further comprising:
adjusting, by the one or more computing devices, an amplitude or a width of the wavelet;
filtering, by the one or more computing devices, the auditory signal using the amplitude or width adjusted wavelet to obtain an extracted amplitude adjusted signal or an extracted width adjusted signal indicating the respiratory abnormality;
determining, by the one or more computing devices, whether a signal amplitude for the extracted amplitude adjusted signal or the extracted width adjusted signal is above the predetermined threshold value;
based on determining the signal amplitude for the extracted amplitude adjusted signal or the extracted width adjusted signal is above the predetermined threshold value, storing, by the one or more computing devices, the extracted amplitude adjusted signal or the extracted width adjusted signal as an instance of the respiratory abnormality; and
based on determining the signal amplitude is below the predetermined threshold value, storing, by the one or more computing devices, the extracted amplitude adjusted signal or the extracted width adjusted signal as an instance indicating no respiratory abnormality.

5. The method of claim 3, further comprising performing, by the one or more computing devices, an inverse wavelet transform on the extracted signal indicating the respiratory abnormality and the extracted signal indicating no respiratory abnormality, to reconstruct the auditory signal.

6. The method of claim 4, further comprising performing, by the one or more computing devices, an inverse wavelet transform on the extracted amplitude adjusted signal or the extracted width adjusted signal indicating the respiratory abnormality, and the extracted amplitude adjusted signal or the extracted width adjusted signal indicating no respiratory abnormality, to reconstruct the auditory signal.

7. The method of claim 3, further comprising:
receiving, by the one or more computing devices, a portion of the auditory signal representing a vesicular sound;
partitioning, by the one or more computing devices, the portion of the auditory signal into segments;
applying, by the one or more computing devices, a transformation to each of the segments to determine a signal envelope;
applying, by the one or more computing devices, a moving average window to the signal envelope to obtain an averaged signal envelope;
identifying, by the one or more computing devices, a point where the averaged signal envelope initially has an amplitude greater than a threshold value;
determining, by the one or more computing devices, a mean value for the amplitude of the averaged signal envelope for a period of time after the point;
determining, by the one or more computing devices, whether the mean value is greater than twice the threshold value; and
based on determining that the mean value is greater than twice the threshold value, identifying, by the one or more computing devices, the point as a start of the respiratory cycle.

8. The method of claim 3, further comprising:
receiving, by one or more computing devices, a portion of the auditory signal representing a vesicular sound;
partitioning, by the one or more computing devices, the auditory signal into segments;
applying, by the one or more computing devices, a transformation to each of the segments to obtain a frequency response of the auditory signal within each of the segments;
summing, by the one or more computing devices, the frequency response across the segments to obtain a summed frequency response;
applying, by the one or more computing devices, an inverse transformation to the summed frequency response to obtain an averaged signal envelope;
identifying, by the one or more computing devices, a point where the averaged signal envelope initially has an amplitude greater than a threshold value;
determining, by the one or more computing devices, a mean value for the amplitude of the averaged signal envelope for a period of time after the point;
determining, by the one or more computing devices, whether the mean value is greater than twice the threshold value; and
based on determining that the mean value is greater than twice the threshold value, identifying, by the one or more computing devices, the point as a start of the respiratory cycle.

9. The method of claim 7, further comprising:
identifying, by the one or more computing devices, a further point where the averaged signal envelope is less than the threshold value;
determining, by the one or more computing devices, a further mean value for the amplitude of the averaged signal envelope for a further period of time prior to the further point;
determining, by the one or more computing devices, whether the further mean value is greater than twice the threshold value; and
based on determining the further mean value is greater than twice the threshold value, identifying, by the one or more computing devices, the further point as an end of the respiratory cycle.

10. The method of claim 9, further comprising:
determining, by the one or more computing devices, a minimum point for the amplitude of the averaged signal envelope between the start of the respiratory cycle and the end of the respiratory cycle; and
identifying, by the one or more computing devices, the minimum point as a start of an expiration event.

11. The method of claim 8, further comprising:
identifying, by the one or more computing devices, a further point where the averaged signal envelope is less than the threshold value;
determining, by the one or more computing devices, a further mean value for the amplitude of the averaged signal envelope for a further period of time prior to the further point;
determining, by the one or more computing devices, whether the further mean value is greater than twice the threshold value; and
based on determining the further mean value is greater than twice the threshold value, identifying, by the one or more computing devices, the further point as an end of the respiratory cycle.

12. The method of claim 11, further comprising:
determining, by the one or more computing devices, a minimum point for the amplitude of the averaged signal envelope between the start of the respiratory cycle and the end of the respiratory cycle; and
identifying, by the one or more computing devices, the minimum point as a start of an expiration event.

13. The method of claim 3, further comprising:
receiving, by the one or more computing devices, a portion of the auditory signal representing respiratory abnormality sounds;
determining, by the one or more computing devices, whether an amplitude for the portion of the auditory signal is above an inspiration threshold; and
based on determining the amplitude is above the inspiration threshold, identifying, by the one or more computing devices, an instance of a respiratory abnormality.

14. The method of claim 13, further comprising:
determining, by the one or more computing devices, whether an amplitude for the portion of the auditory signal is above an expiration threshold; and
based on determining the amplitude is above the expiration threshold, identifying, by the one or more computing devices, a further instance of the respiratory abnormality.

15. A digital stethoscope for de-noising an auditory signal comprising:
a memory configured to store instructions;
one or more processors coupled to the memory configured to process the stored instructions to:
partition an auditory spectrogram representing the auditory signal into a plurality of windows of equal length timeframes, wherein each of the windows indicates a frequency response of the auditory signal within each of the timeframes; and
process each of the windows using a neural network trained to remove unwanted noise signals from the auditory signal, wherein the processing comprises:
(i) identifying an odd number of consecutive windows,
(ii) identifying a middle window from the odd number of consecutive windows, wherein the middle window is a window to have the unwanted noise signals removed,
(iii) identifying an even number of windows preceding the middle window,
(iv) identifying an even number of windows following the middle window,
(v) inputting the middle window, the even number of windows preceding the middle window, and the even number of windows following the middle window into the neural network, and
(vi) computing, using the neural network, a vector representing the auditory signal with the unwanted noise signals removed.

16. The digital stethoscope of claim 15, wherein the one or more processors are further configured to repeat (i)-(vi) for each of the plurality of windows of the auditory spectrogram.

17. The digital stethoscope of claim 15, wherein the one or more processors are further configured to:
filter the auditory signal by performing a wavelet transform, wherein the wavelet transform utilizes a wavelet representing a sound indicating a respiratory abnormality, the wavelet transform to extract a signal from the auditory signal indicating the respiratory abnormality;
determine whether a signal amplitude for the extracted signal is above a predetermined threshold value;
based on determining the signal amplitude is above the predetermined threshold value, store the extracted signal as an instance of the respiratory abnormality; and
based on determining the signal amplitude is below the predetermined threshold value, store the extracted signal as an instance indicating no respiratory abnormality.

18. The digital stethoscope of claim 17, wherein the one or more processors are further configured to:
adjust an amplitude or a width of the wavelet;
filter the auditory signal using the amplitude or width adjusted wavelet to obtain an extracted amplitude adjusted signal or an extracted width adjusted signal indicating the respiratory abnormality;
determine whether a signal amplitude for the extracted amplitude adjusted signal or the extracted width adjusted signal is above the predetermined threshold value;
based on determining the signal amplitude for the extracted amplitude adjusted signal or the extracted width adjusted signal is above the predetermined threshold value, store the extracted amplitude adjusted signal or the extracted width adjusted signal as an instance of the respiratory abnormality; and
based on determining the signal amplitude is below the predetermined threshold value, store the extracted amplitude adjusted signal or the extracted width adjusted signal as an instance indicating no respiratory abnormality.

19. The digital stethoscope of claim 17, wherein the one or more processors are further configured to perform an inverse wavelet transform on the extracted signal indicating the respiratory abnormality and the extracted signal indicating no respiratory abnormality, to reconstruct the auditory signal.

20. The digital stethoscope of claim 18, wherein the one or more processors are further configured perform an inverse wavelet transform on the extracted amplitude adjusted signal or the extracted width adjusted signal indicating the respiratory abnormality, and the extracted amplitude adjusted signal or the extracted width adjusted signal indicating no respiratory abnormality, to reconstruct the auditory signal.
